(19) United States
(12) Patent Application Publication
(10) Pub. No.: US 2018/0256616 A1
(43) Pub. Date: Sep. 13, 2018

(54) Compounds, Compositions and Methods for Prevention or Treatment of Liver and Lipid-Related Conditions

(71) Applicant: Sivanaray, Inc., Corona, CA (US)

(72) Inventor: Nallagounder Kuppusamy, Corona, CA (US)

(21) Appl. No.: 15/748,130

(22) PCT Filed: Jul. 28, 2016
(86) PCT No.: PCT/US2016/044556

§ 371 Date: Jan. 26, 2018

Related U.S. Application Data
(60) Provisional application No. 62/199,485, filed on Jul. 31, 2015.

(51) Int. Cl. A61K 31/7004; A61K 31/7012; A61P 1/16; A61P 3/06

(52) U.S. Cl. CPC A61K 31/7004; A61P 3/06; A61P 1/16; A61K 31/7012

(57) ABSTRACT

The inventive subject matter provides compositions and methods for reducing serum levels of at least one biomarker associated with a lipid or liver-related condition. Preferred compositions include a set of actives that consists essentially of at least two saccharides selected from galacturonic acid, galactose, arabinose, rhamnose, glucose, xylose, and mannose.

This application claims priority to U.S. provisional application with the Ser. No. 62/199,485, which was filed on Jul. 31, 2015. This and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### FIELD OF THE INVENTION

The field of the invention is compounds, compositions and methods for prevention or treatment of liver and lipid-related conditions.

### BACKGROUND

The background description includes information that may be useful in understanding the present invention. It is not an admission that any of the information provided herein is prior art or relevant to the presently claimed invention, or that any publication specifically or implicitly referenced is prior art.

The liver is one of the largest and most important organs in the body, playing a vital role in the removal of waste products from the blood, distribution and storage essential nutrients, and breakdown of harmful substances such as alcohol and toxic chemicals. Given the many vital functions performed by the liver, conditions affecting the liver can have devastating effects, whether hereditary or caused by pathogens, exposure to toxins, or lifestyle conditions such as diet, alcohol consumption and caloric intake.

For example, nonalcoholic fatty liver disease (NAFLD) and nonalcoholic steatohepatitis (NASH) are common liver disorders associated with over-accumulation of lipids, which can be marked by inflammation and scarring in the liver. While most people with NAFLD or NASH are asymptomatic, these and other liver diseases can ultimately result in cirrhosis of the liver, hepatic encephalopathy, acute kidney injury, or even death. Some lipid and liver-related diseases have also been linked to insulin resistance, diabetes, and cardiovascular disorders.

As a healthy liver and lipid profile are crucial to the health and well being of complex organisms, there is a significant need for effective compounds, compositions and methods for preventing and treating lipid and liver related disorders, especially those showing minimum toxicity and adverse effects.

There are several known efforts that have included the use of plant or fruit-derived polysaccharides and specific modified forms thereof to treat NASH and other lipid or liver-related conditions. For example, Galectin Therapeutics Inc. has contemplated the use of modified polysaccharides synthesized from pectin for the treatment of NASH (See e.g., U.S. Pat. No. 8,658,787). As another example, Preventative Effects of Jujube Polysaccharides on Fructose-Induced Insulin Resistance and Dyslipidemia in Mice (Zhao, et al.) has investigated whether polysaccharides derived from Zizyphys jujube cv. Shaanbeitanzao could alleviate high fructose-induced insulin resistance and dyslipidemia in mice.

Pectin and Jujube derived polysaccharides discussed in the '787 patent and Zhao include various types of sugars, and are soluble dietary fibers. While these compounds have been reported as beneficial in providing some liver protective qualities, they tend to have a laxative effect, causing diarrhea, nausea or bloating in some cases. Furthermore, the amounts of these polymers required to product a desired effect can be inconvenient in terms of both volume and laxative effect.

Thus, there is still a need for improved and effective compounds, compositions and methods for preventing and treating lipid and liver related disorders.

### SUMMARY OF THE INVENTION

Applicant surprisingly discovered that selected components of naturally derived polysaccharides are highly effective for treating various lipid and liver related disorders. Compounds and compositions of the inventive subject matter preferably include isolated and purified forms of the selected effective components with improved bioavailability and reduced adverse effects compared to known polysaccharides. Specific saccharide components have been found to be particularly effective when used in specified ratios and optionally in synergistic amounts with respect to the condition(s) to be treated.

In one aspect, compositions including combinations of two or more isolated and purified saccharides can effectively reduce serum levels of at least one biomarker associated with a lipid or liver-related condition when administered (e.g., orally) for a specified minimum amount of time. Contemplated compositions are especially effective when administered to a person having steatohepatitis or type 2 diabetes, or a person that is overweight (having a body mass index (BMI) of greater than 25, more preferably greater than 30). It is contemplated that the specified minimum amount of time can be at least 7 days, at least 14 days, at least 28 days, at least 42 days, at least 56 days, between 1-70 days, between 14-42 days, between 14-28 days, or any other suitable time.

For example, a composition can include a set of active components that is present in a therapeutically effective amount to lower at least one of a HOMA-IR value, a total cholesterol serum level, fat accumulation in liver, a liver oxidative stress marker level (e.g., MDA), a serum urea level, a serum free fatty acid level, a serum triglyceride level, a serum ALT level, a serum ALP level, a serum hs-CRP level, a serum PTX3 level, a serum leptin level, a serum MCP-1 level, a serum insulin level, and a serum LDL level (or any subset thereof). The inventive subject matter also provides methods for lowering a level of at least one of these biomarkers by formulating or obtaining a composition as set forth herein, and administering the composition for a period of at least 2 weeks, more preferably at least 4 weeks, and most preferably at least 6 weeks. Viewed from a different perspective, compositions of the inventive subject matter can include a set of active components that is present in a therapeutically effective amount to treat a condition associated with, or lower at least one of a HOMA-IR value, a total cholesterol serum level, fat accumulation in liver, a liver oxidative stress marker level (e.g., MDA), a serum urea level, a serum free fatty acid level, a serum triglyceride level, a serum ALT level, a serum ALP level, a serum hs-CRP level, a serum PTX3 level, a serum leptin level, a serum MCP-1 level, a serum insulin level, and a serum LDL level, when orally or parenterally administered for a period of at least 2 weeks, more preferably at least 4 weeks, and most preferably at least 6 weeks.

In some preferred embodiments, the set of active components comprises at least two, at least three, at least four, at least five, at least six, or even all seven of the saccharides selected from: galacturonic acid, galactose, arabinose, rhamnose, glucose, xylose, and mannose. Most preferably, each of the at least two saccharides are present in isolated and purified monosaccharide forms, optionally in synergistic quantities with respect to lowering the level of at least one of the biomarkers discussed above, treating a condition associated with at least one of the biomarkers, weight loss, or fat accumulation in the liver.

In some compositions, the set of active components for lowering a level of at least one biomarker in serum or liver sample (e.g., HOMA-IR, total cholesterol, fat accumulation in liver, MDA, urea, free fatty acids, triglycerides, ALT, ALP, hs-CRP, PTX-3, leptin, MCP-1, insulin, LDL) can essentially consist of at least two, at least three, at least four, at least five, at least six, or even all seven of the isolated and purified saccharides. For example, the isolated and purified saccharides selected from galacturonic acid, galactose, arabinose, rhamnose, glucose, xylose and mannose can comprise at least 85%, more preferably at least 90%, and even more preferably at least 95% of the set of active components in the composition.

It should be appreciated that the compositions contemplated herein can comprise active components other than the saccharides listed herein, so long as the saccharides in the set of actives alone are effective to at least one of (a) cause weight loss, (b) reduce a level of at least one biomarker associated with lipid or liver-related conditions, (c) reduce fat accumulation in the liver, and (d) treat a condition associated with the biomarker when administered in accordance with a dosing schedule (e.g., between 1-500 mg/kg body weight per day for at least 2 weeks, between 1-250 mg/kg body weight per day for at least 6 weeks, between 1-100 mg/kg body weight per day for at least 4 weeks, between 1-40 mg/kg body weight per day for at least 6 weeks, between 2-20 mg/kg body weight per day for at least 6 weeks). It should also be appreciated that compositions of the inventive subject matter could be administered with other compositions having active ingredients other than the saccharides listed herein. Additional ingredients are considered supplemental or additive, and may or may not be present in synergistic quantities with respect to lowering serum levels of specified biomarkers.

The isolated and purified saccharides in contemplated compositions can have a purity of between 40-100% (GC), for example between 70-100%, between 80-100%, between 90-100%, between 95-100%, and most preferably between 98-100%. For purposes of the studies discussed herein, each saccharide was isolated and had a purity of at least 90% (GC). Additionally, the isolated and purified saccharides in contemplated compositions can be present in any suitable molar or weight ratio. In some preferred embodiments, each saccharide will be present in a molar ratio that is no greater than 20:1, more preferably no greater than 10:1, with respect to each of the other individual saccharides in the composition.

For example, a composition of the inventive subject matter can comprise a set of active components that consists essentially of isolated and purified galacturonic acid, isolated and purified galactose and isolated and purified arabinose, wherein the set of active components are effective, when orally or otherwise administered, to lower at least one of the HOMA-IR value, the total cholesterol serum level, fat accumulation in liver, the liver oxidative stress marker level, the serum urea level, the serum free fatty acid level, the triglyceride serum level, the ALT serum level, the ALP serum level, the hs-CRP serum level, the PTX3 serum level, the leptin serum level, the MCP-1 serum level, the insulin serum level, and the LDL serum level (or any subset thereof). The molar ratio of galactose to galacturonic acid can be between 1:15 and 15:1, more preferably between 1:2 and 5:1, and even more preferably between 1:1 and 3:1. The molar ratio of arabinose to galacturonic acid can be between 1:15 and 15:1, between 1:10 and 10:1, more preferably between 2:1 and 10:1, and even more preferably between 4:1 and 8:1, or between 5:1 and 7:1.

As another example, a composition of the inventive subject matter can comprise a set of active components that consists essentially of isolated and purified galacturonic acid and isolated and purified galactose, wherein the set of active components are effective, when orally administered, to lower at least one of the HOMA-IR value, the total cholesterol serum level, fat accumulation in liver, the liver oxidative stress marker level, the serum urea level, the serum free fatty acid level, the triglyceride serum level, the ALT serum level, the ALP serum level, the hs-CRP serum level, the PTX3 serum level, the leptin serum level, the MCP-1 serum level, the insulin serum level, and the LDL serum level. The molar ratio of galactose to galacturonic acid can be between 1:20 and 20:1, between 1:15 and 15:1, more preferably between 1:10 and 10:1, and even more preferably between 1:2 and 5:1.

As the compositions of the inventive subject matter have been found to be non-toxic at 1,000 mg/kg/day when administered to an animal model, it is contemplated that a wide range of doses of the active components could be administered to treat various conditions, optionally with one or more other active ingredients, and without significant adverse effects (e.g., laxative effects, aging effects).

In some aspects, contemplated compositions can comprise a set of active components as described above, in amounts effective to treat a condition (e.g., disorders, diseases, symptoms) associated with at least one of a HOMA-IR value, a total cholesterol serum level, fat accumulation in liver, a liver oxidative stress marker level (e.g., MDA), a serum urea level, a serum free fatty acid level, a serum triglyceride level, a serum ALT level, a serum ALP level, a serum hs-CRP level, a serum PTX3 level, a serum leptin level, a serum MCP-1 level, a serum insulin level, and a serum LDL level (or any subset thereof).

Similarly, methods for treating conditions associated with at least one of triglycerides, ALT (SGPT) and LDL levels in serum are contemplated, as well as methods for reducing fat accumulation in a liver of a person, and methods for treating conditions associated with, or lowering at least one of a HOMA-IR value, a total cholesterol serum level, a liver oxidative stress marker level (e.g., MDA), a serum urea level, a serum free fatty acid level, a serum triglyceride level, a serum ALT level, a serum ALP level, a serum hs-CRP level, a serum PTX3 level, a serum leptin level, a serum MCP-1 level, a serum insulin level, and a serum LDL level. Suitable methods can comprise formulating or obtaining a composition comprising a set of active components in a therapeutically effective amount, and administering to a subject in need thereof an effective dose of the composition (e.g., between 5-50 mg/kg body weight per day, between 10-25 mg/kg body weight per day) for a period of at least 2 weeks, at least 4 weeks, or at least 6 weeks. Preferably, the administration will result in an at least 10% reduction, more preferably an at least 20% reduction, an at least 25% reduction, an at least 40% reduction, or even an at least 50% reduction in a value or serum/liver tissue level of one, two, three, four, five or even more (e.g., all) of the biomarkers (e.g., LDL, triglycerides, ALT, HOMA-IR, total cholesterol, fat accumulation in liver, MDA, urea, free fatty acids, ALP, hs-CRP, PTX3, leptin, MCP-1, insulin).

Conditions that can be effectively treated using compositions and methods of the inventive subject matter include, among other conditions, obesity (which has been associated with elevated serum ALT, ALP, triglycerides, PTX-3, leptin, or hs-CRP levels), diabetes (which has been associated with elevated serum PTX-3, leptin, or MCP-1 levels), liver conditions, such as hepatitis, alcoholic hepatitis, autoimmune hepatitis, toxic hepatitis, nonalcoholic fatty liver disease, alcoholic or non-alcoholic steatohepatitis, cirrhosis, hypothyroidism (which have been associated with elevated HOMA-IR value, or elevated ALT and ALP serum levels), diabetes, hypothyroidism, increased risk of heart disease, and kidney disease (which have been associated with elevated levels of triglycerides), inflammation, heart attacks and strokes, liver disorders, and cardiovascular disorders (which have been associated with elevated serum hs-CRP levels), Alzheimer's disease, kidney disorders, lung disorders, liver disorders, pancreas disorders, cardiovascular disorders, and diabetes (which have been associated with elevated serum PTX-3 and leptin levels), alcoholic liver disease or injury, obesity, kidney disorders, liver disorders, pancreas disorders, cardiovascular disorders, and even cancer (which have been associated with elevated MCP-1 levels), insulin resistance (e.g., type 2 diabetes), insulinomas, fructose or galactose intolerance, cushing syndrome, acromegaly (which have been associated with elevated serum insulin levels), increased risk of heart disease, heart disease (which have been associated with high LDL cholesterol levels), hypertension, atherosclerosis, dyslipidemia, kidney fibrosis, liver fibrosis, lung fibrosis, heart fibrosis, other inflammatory or autoimmune disorders, neoplastic conditions, ascites, gall stones, syndrome X, and primary biliary sclerosis.

The inventive subject matter also comprises the use of a set of active components as described above, in a composition to treat a condition associated with at least one of a HOMA-IR value, a total cholesterol serum level, fat accumulation in liver, a liver oxidative stress marker level, a serum urea level, a serum free fatty acid level, a serum triglyceride level, a serum ALT level, a serum ALP level, a serum hs-CRP level, a serum PTX3 level, a serum leptin level, a serum MCP-1 level, a serum insulin level, and a serum LDL level (for example to treat a condition associated with a HOMA-IR value; to treat a condition associated with a total cholesterol serum level; to treat a condition associated with fat accumulation in liver; to treat a condition associated with a liver oxidative stress marker; to treat a condition associated with a serum urea level; to treat a condition associated with a serum free fatty acid level; to treat a condition associated with a triglyceride level; to treat a condition associated with a seru ALT level; to treat a condition associated with a serum ALP level; to treat a condition associated with a serum hs-CRP level; to treat a condition associated with a serum PTX-3 level; to treat a condition associated with a serum leptin level; to treat a condition associated with a serum MCP-1 level; to treat a condition associated with a serum insulin level; to treat a condition associated with a serum LDL level).

In another aspect, the inventive subject matter contemplates the use of a set of active components as described above in a composition to reduce at least one of a HOMA-IR value, a total cholesterol serum level, fat accumulation in liver, a liver oxidative stress marker level, a serum urea level, a serum free fatty acid level, a serum triglyceride level, a serum ALT level, a serum ALP level, a serum hs-CRP level, a serum PTX3 level, a serum leptin level, a serum MCP-1 level, a serum insulin level, and a serum LDL level (for example, to reduce a HOMA-IR value; to reduce a total cholesterol serum level; to reduce fat accumulation in the liver; to reduce a liver oxidative stress marker level (e.g., MDA); to reduce a serum urea level, to reduce a serum free fatty acid level; to reduce a triglyceride serum level; to reduce a ALT serum level; to reduce a ALP serum level; to reduce a hs-CRP serum level; to reduce a PTX-3 serum level; to reduce a leptin serum level; to reduce a MCP-1 serum level; to reduce a insulin serum level; to reduce a LDL serum level).

Still further, the inventive subject matter contemplates the use of at least three isolated and purified saccharides selected from: galacturonic acid, galactose, arabinose, rhamnose, glucose, xylose, and mannose in the manufacture of a therapeutic drug for the treatment of a condition associated with, or lowering of, at least one a HOMA-IR value, a total cholesterol serum level, fat accumulation in liver, a liver oxidative stress marker level, a serum urea level, a serum free fatty acid level, a serum triglyceride level, a serum ALT level, a serum ALP level, a serum hs-CRP level, a serum PTX3 level, a serum leptin level, a serum MCP-1 level, a serum insulin level, and a serum LDL level (or any subset thereof).

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1A illustrates a comparison of serum biomarker levels and body weight in an animal control group and an animal group fed a high fat diet.

FIG. 1B illustrates a comparison of glucose levels measured following the first administration of the formulation of Study 1.

FIGS. 2A-2B show a comparison of plasma biochemical parameters of the different groups of animals studied at the end of the first, second, third and fourth weeks of the treatment period of Study 1.

FIG. 3 shows the lack of differences in feed and water intake, body weight change and coagulation time between Groups 1 and 2 during Study 1.

FIG. 4 shows the results of liver enzyme tests in serum performed in Study 1 after the four weeks of treatment.

FIG. 5 shows a comparison of the morphometry of the liver and kidney in animals of the different groups in Study 1.

FIG. 6 shows a comparison of biomarkers of oxidative stress in Study 1.

FIGS. 7A-7E illustrate stained sections of liver biopsies from animals of several groups of Study 1.

FIGS. 8A-8C illustrate the serum levels of several biomarkers in the animals during the ten week period prior to administration of a VISIVABRM formulation in Study 2.

FIGS. 9A-9C show a comparison of plasma biochemical parameters of the different groups of animals studied at the end of the second and sixth weeks of the treatment period in Study 2.

FIG. 10A-10D show plasma biochemical parameters of the different groups of animals studied at the end of the sixth week of the treatment period in Study 2.

FIG. 11 shows a comparison of biomarkers of oxidative stress in Study 2.

FIG. 12A shows the ratio of liver weight to body weight of animals in the different groups at the end of Study 2.

FIG. 12B shows the change in body weight of animals in the different groups at the end of Study 2.

FIG. 13 shows images taken of liver samples of animals in the different groups in Study 2.

FIGS. 14A-14E show representative photomicrographs of liver sections of three animals randomly selected from each of control group, HFFrD group (NASH), and treated groups of Study 2.

FIGS. 15A-15B show a comparison of plasma biochemical parameters of the different groups of animals studied at different times during Study 3.

### DETAILED DESCRIPTION

The inventive subject matter provides compositions and methods for lowering, and treating a condition associated with, a HOMA-IR value, a total cholesterol serum level, fat accumulation in liver, a liver oxidative stress marker level, a serum urea level, a serum free fatty acid level, a serum triglyceride level, a serum ALT level, a serum ALP level, a serum hs-CRP level, a serum PTX3 level, a serum leptin level, a serum MCP-1 level, a serum insulin level, a serum LDL level, or any combinations thereof.

For example, in one study showing the efficacy of compositions and methods of the inventive subject matter as further discussed below, animals were fed a high fat diet for thirty days and then injected with streptozotocin to induce type 2 diabetes. While continuing the high fat diet, various amounts of a formulation comprising isolated and purified forms of galacturonic acid, galactose, arabinose, rhamnose, glucose, xylose and mannose monosaccharides were orally administered to the animals for a period of four weeks. The formulation was found to significantly reduce the level of several indicators of liver and lipid-related disorders, including triglycerides, low-density lipoproteins (LDL), malondialdehyde (MDA), alanine transaminase (ALT) and alkaline phosphatase (ALP). Additionally, animals treated with the formulation showed significant weight loss and histologic improvement in steatosis.

However, it should be appreciated that effective compositions do not need to include each of galacturonic acid, galactose, arabinose, rhamnose, glucose, xylose and mannose. Indeed efficacy has been shown with various combinations of these saccharides in various molar ratios, and the selection of saccharides may be driven by the result desired and the condition to be treated, for example, as guided by the examples shown herein.

The isolated and purified saccharides can have any suitable purity, but will preferably have a purity of between 40-100% (GC), for example between 70-100%, between 80-100%, between 90-100%, between 95-100%, and most preferably between 98-100%. Additionally, while the sugars studied were naturally occurring monomers—naturally occurring and synthetically produced monomers, dimers, oligomers (3-10 monosaccharide units), and polymers (more than 10 monosaccharide units) are also contemplated. For example, a saccharide may be obtained from a natural source such as a plant, fruit or vegetable, and optionally fragmented by acid, alkaline or catalytic hydrolysis, enzymatic digestion, oxidative lysis or radiative lysis.

Where the saccharides are not monomers, both heteromeric and homomeric saccharides are contemplated. It should be appreciated that the isolated and purified saccharides can comprise L-isomers, D-isomers, or a mixture of L-isomers and D-isomers. In some embodiments, the saccharides can comprise alpha linkages, beta linkages, or a combination of alpha and beta linkages. In some embodiments, one or more of the saccharides may be esterified, methylated, acetylated, amidated, or otherwise modified, preferably with a nutritionally acceptable component. Additionally, the sugars can be present in open-chain or ring form (e.g., furanoses, pyranoses). Moreover, all neutraceutical and pharmaceutically acceptable salts and pro-drugs are expressly contemplated herein.

In some embodiments, two or more of the same or different saccharides, preferably monomers, can be combined and bound together by glycosidic linkages to make a backbone, oligomeric or polymeric structure. However, other linkages are also deemed suitable, particularly where the linkage is nutritionally or pharmaceutically acceptable (e.g., polylactides, PEG, glycols, diesters). For example, isolated and purified galacturonic acid, isolated and purified galactose and isolated and purified arabinose monomers could be bound together by alpha or beta glycosidic linkages to form an hetero or homo oligomeric or polymeric structure wherein the molar ratio of galactose to galacturonic acid can be between 1:15 and 15:1, between 1:10 and 10:1, more preferably between 1:2 and 5:1, and even more preferably between 1:1 and 3:1, and the molar ratio of arabino se to galacturonic acid can be between 1:15 and 15:1, between 1:10 and 10:1, more preferably between 2:1 and 10:1, and even more preferably between 4:1 and 8:1, or between 5:1 and 7:1. Preferably, the oligomeric or polymeric structure will consist essentially of (i.e., be at least 85% composed of) galacturonic acid, galactose, arabinose, xylose, glucose, mannose, rhamnose, or any subset or combinations thereof. The so formed oligomer or polymer can have between 3 and 10, between 10 and 100, or even between 100 and 1,000 (or even more) monomers connected in linear or branched forms.

The isolated and purified saccharides in contemplated compositions can be present in any suitable molar or weight ratio. In some preferred embodiments, each saccharide will be present in a molar ratio that is no greater than 20:1, more preferably no greater than 10:1, with respect to each of the other individual saccharides in the composition. As some non-limiting examples, the isolated and purified saccharides in contemplated compositions can e present in any of the molar ratios shown in Table 1.

TABLE 1

| Saccharide | Moles | Moles | Moles | Moles | Moles | Moles | Moles | Moles | Moles |
|---|---|---|---|---|---|---|---|---|---|
| Galacturonic | 1 | 1 | 1 | 1 | 1 | 1 | 1 | 1 | 1 |
| Acid | | | | | | | | | |
| Galactose | .2-1.5 | .2.-7 | 1-3 | 1-3 | .1-10 | .1-10 | .1-10 | .05-20 | .05-20 |
| Arabinose | .3-4.5 | .3-.8 | 1-5 | 4-8 | .1-10 | .1-10 | | .05-20 | |
| Rhamnose | .05-.5 | .05-.1 | .1-.6 | .5-1 | .1-10 | | | | |
| Glucose | .05-1 | | | 1-2 | .1-10 | | | | |
| Xylose | .5-4 | | | 4-6 | .1-10 | | | | |
| Mannose | .5-1.5 | | | .5-3.5 | .1-10 | | | | |

Heretofore, it was unknown and unexpected that specific monomer components of naturally derived polysaccharides could also be highly effective for treating various lipid and liver related disorders. Existing literature was replete with examples that emphasized the liver benefitting effects resulting from the use of only polysaccharides. Applicant was surprisingly able to identify the effective monomer components, and combine them in effective (and in certain cases synergistic) ratios to improve the biological effect of the effective components relative to known polysaccharides.

Even upon unexpectedly identifying the effectiveness of various combinations of the effective monomers, it was unexpected that providing the effective components in monomeric form would show the same, similar or improved efficacy as administering known polysaccharide compounds. This result was as unexpected as it would have been to administer select individual amino acids present in insulin, and expect to achieve the same or improved efficacy as insulin.

With respect to the amount of contemplated set of actives in the composition, it should be recognized that the particular quantity will typically depend on the specific formulation, active ingredient, and desired purpose. Therefore, it should be recognized that the amount of actives in contemplated compositions will vary significantly. However, it is generally preferred that the set of actives is present in a minimum amount effective to deliver a therapeutic effect or to be achieved in vitro or in vivo.

The compositions according to the invention may be delivered in a therapeutically effective amount. The term “therapeutically effective amount” refers to the amount of the compound or composition that will elicit a biological or medical response of a tissue, system, animal or human that is being sought (e.g., reduction in weight; reduction in a HOMA-IR value; reduction in a total cholesterol serum level; reduction in fat accumulation in the liver; reduction in a liver oxidative stress marker level (e.g., MDA); reduction in serum urea level, reduction in serum free fatty acid level; reduction in triglyceride serum level; reduction in ALT serum level; reduction in ALP serum level; reduction in hs-CRP serum level; reduction in PTX-3 serum level; reduction in leptin serum level; reduction in MCP-1 serum level; reduction in insulin serum level; reduction in LDL serum level; reduction in at least one of the HOMA-IR value, a total cholesterol serum level, fat accumulation in liver, the liver oxidative stress marker level, the serum urea level, the serum free fatty acid level, the triglyceride serum level, the ALT serum level, the ALP serum level, the hs-CRP serum level, the PTX3 serum level, the leptin serum level, the MCP-1 serum level, the insulin serum level, and the LDL serum level; improvement in liver steatosis).

The optimum therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typically, the dose is normally from 0.01 to 500 mg/kg of body weight per day, more preferably from 0.1 to 100 mg, from 0.1 to 50 mg/kg/day, or from 3.5 to 50 mg/kg/day. While the formulations described herein were administered to animals at between 50-1,000 mg/kg body weight per day, the person skilled in the arts could calculate the human effective dosage from known Kₘ factors. Depending on the animal models used, the human effective dosage will typically be between 5-35%, more preferably 7-15% in humans of the animal effective dosage.

The administration of the suitable dose can be administered once per day, or can be spread out over the course of a day. For example, an effective dose of the composition can be divided and separately packaged as two to five capsules, tablets, powders or oral dissolve strips, and separately administered two to five times a day. Alternate day dosing or dosing once every several days may also be utilized. Depending on the particular use and structure, it is contemplated that the set of actives according to the inventive subject matter are present in the composition in an amount between 1 microgram to 1000 milligram, more typically between 10 microgram to 500 milligram, and most typically between 10 mg to 250 mg per single dosage unit. Thus, concentrations of contemplated compounds in vivo or in vitro may be between 0.1 nM and 100 microM, more typically between 1 nM and 50 microM, and most typically between 10 nM and 10 microM.

The compositions according to the inventive subject matter may be administered using various routes, but is preferably administered orally in any orally acceptable dosage form including, but not limited to, capsules, powders, tablets, troches, elixirs, suspensions, syrups, wafers, chewing gums, aqueous suspensions or solutions.

The pharmaceutical preparations can be made following the conventional techniques of pharmacy involving milling, mixing, granulation, and compressing, when necessary, for tablet forms; or milling, mixing and filling for hard gelatin capsule forms. When the dosage unit form is a capsule, it may additionally contain a pharmaceutically acceptable carrier, such as a liquid carrier (e.g., a fatty oil). Other dosage unit forms may contain other various materials which modify the physical form of the dosage unit, such as, for example, a coating. Thus, tablets or pills may be coated with sugar, shellac, or other enteric coating agents. A liquid or syrup may contain, in addition to the active ingredients, sucrose as a sweetening agent and certain preservatives, dyes and colorings and flavors. Materials used in preparing these various compositions should be pharmaceutically or veterinarally pure and non-toxic in the amounts used. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation.

Although oral compositions may be preferred, all commercially suitable routes of administration are contemplated, including oral, parenteral, inhalation, topical, rectal, nasal, or via an implanted reservoir, wherein the term “parenteral” as used herein includes subcutaneous, intravenous, intramuscular, intraarticular, intrasynovial, intrathecal, intrahepatic, intralesional, and intracranial administration (typically injection or infusion).

For therapeutic or prophylactic purposes, contemplated compounds are ordinarily combined with one or more excipients appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, or polyvinyl alcohol, and then tableted or encapsulated for convenient administration. Such capsules or tablets may contain a controlled-release formulation as may be provided in a dispersion of active compound in hydroxypropylmethyl cellulose.

Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, or various buffers. Other excipients and modes of administration are well and widely known in the pharmaceutical art.

For the purposes of parenteral therapeutic administration, the active ingredient may be incorporated into a solution or suspension. The solutions or suspensions may also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

The pharmaceutical forms suitable for injectable use include sterile solutions, dispersions, emulsions, and sterile powders. The final form should be stable under conditions of manufacture and storage. Furthermore, the final pharmaceutical form should be protected against contamination and should, therefore, be able to inhibit the growth of microorganisms such as bacteria or fungi. A single intravenous or intraperitoneal dose can be administered. Alternatively, a slow long-term infusion or multiple short-term daily infusions may be utilized, typically lasting from 1 to 8 days.

Sterile, injectable solutions may be prepared by incorporating a compound or set of actives in the required amount into one or more appropriate solvents to which other ingredients, listed above or known to those skilled in the art, may be added as required. Sterile injectable solutions may be prepared by incorporating the compound in the required amount in the appropriate solvent with various other ingredients as required. Sterilizing procedures, such as filtration, may then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

Suitable pharmaceutical carriers include sterile water; saline, condensation products of castor oil and ethylene oxide combining about 30 to about 35 moles of ethylene oxide per mole of castor oil; liquid acid; lower alkanols; oils such as corn oil; peanut oil, sesame oil and the like, with emulsifiers such as mono- or di-glyceride of a fatty acid, or a phosphatide, e.g., lecithin, and the like; glycols; polyalkylene glycols; aqueous media in the presence of a suspending agent, for example, sodium carboxymethylcellulose; sodium alginate; poly(vinylpyrolidone) ; and the like, alone, or with suitable dispensing agents such as lecithin; polyoxyethylene stearate; and the like. The carrier may also contain adjuvants such as preserving stabilizing, wetting, emulsifying agents and the like together with the penetration enhancer. In all cases, the final form, as noted, must be sterile and should also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

While compositions are preferably in pharmaceutical form, nutraceuticals such as dietary supplements, nutritional supplements and medical foods are also contemplated. The nutraceuticals can optionally be used in combination with foods, beverages, spices, condiments, salad dressings and any other goods where soluble starches or fibers are used.

Depending on the particular purpose, it should also be recognized that contemplated compounds, compositions or sets of active compounds may be combined (in vivo, or in a therapeutic formulation or administration regimen) with at least one other therapeutically active agent to additively or synergistically provide a therapeutic or prophylactic effect. Concentrations of the other therapeutically active ingredients are typically at or preferably below those recommended for stand-alone administration, however, higher concentrations are also deemed suitable for use herein. Additional ingredients are considered supplemental or additive, and may or may not be present in synergistic quantities with respect to weight loss, treating a condition associated with, or lowering, a HOMA-IR value, a total cholesterol serum level, fat accumulation in liver, a liver oxidative stress marker level, a serum urea level, a serum free fatty acid level, a serum triglyceride level, a serum ALT level, a serum ALP level, a serum hs-CRP level, a serum PTX3 level, a serum leptin level, a serum MCP-1 level, a serum insulin level, and a serum LDL level.

The additional ingredients could include, for example, anti-inflammatory drugs, especially non-NSAIDs such as cannabidiol, betaine, turmeric extract, boswellia serrata extract, bromelain extract, ginger extract, or red rice yeast.

The effectiveness of Applicant's compositions and methods have been supported by several experimental studies performed on animals, including control groups, animals with induced non-alcoholic steatohepatitis (NASH), and animals with induced type 2 diabetes (T2DM), as set forth below in Studies 1-3. In this series of studies, various combinations of isolated and purified monosaccharides were administered orally to these animals.

### Study 1 (AMUDAM Study)

In Applicant's first Study of the disclosed compositions, a formulation of the inventive subject matter (hereinafter “AMUDAM”) was administered to selected groups of rats in different dosage amounts. During the first 4 weeks of the study, the control group rats (n=15) were fed a diet of normal pellets, and the high fat diet (HFD) group rats (n=32) was fed HFD pellets.

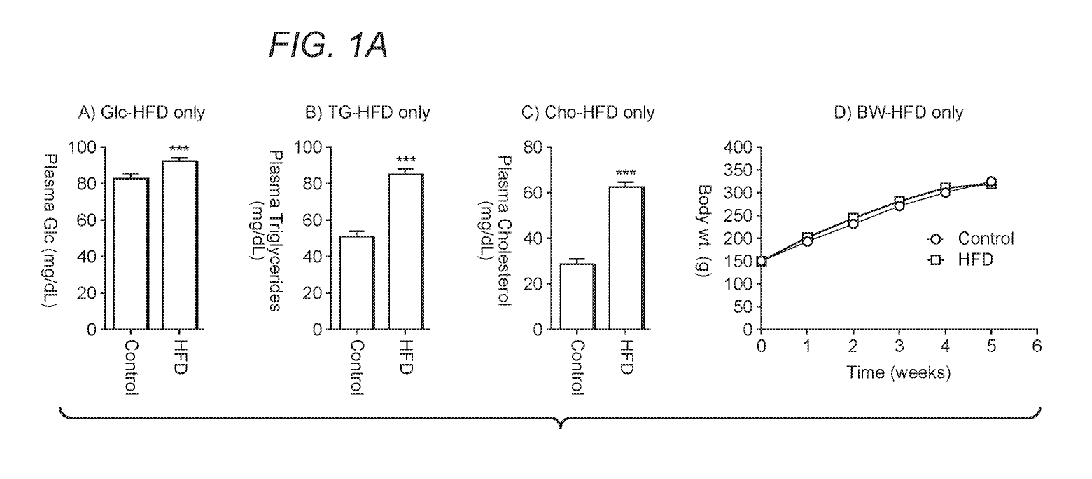

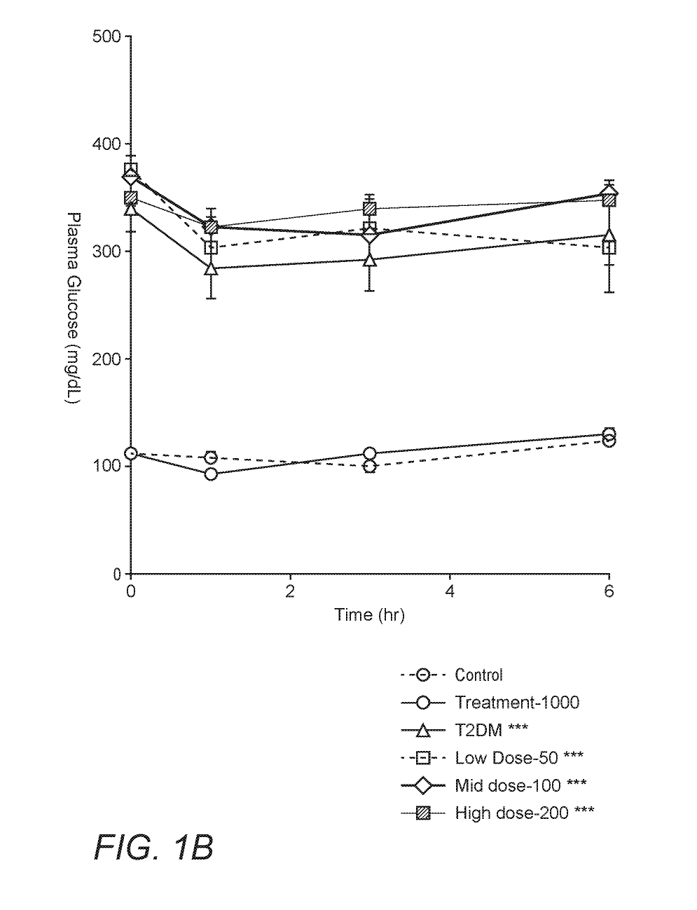

After the first 4 weeks of being fed normal or HFD pellets, serum glucose, triglycerides and cholesterol levels were determined. As shown in Table 2 and FIG. 1A, rats that were fed HFD pellets showed significant increase in glucose, triglycerides and cholesterol levels, but the weight increase in both groups was substantially the same.

TABLE 2

| | Glucose | Triglyceride | Cholesterol | |
|---|---|---|---|---|
| | (mg/dL) | (mg/dL) | (mg/dL) | Weight |
| Control | app. 80 | app. 50 | app. 30 | Increased from |
| | | | | app. 150 g-app. |
| | | | | 300 g |
| HFD | 90 | 80 | 60 | Increased from |
| | | | | app. 150 g-app. |
| | | | | 300 g |

After the first 4 weeks, the HFD group was injected with streptozotocin (STZ) to induce type 2 diabetes. The control group was split into two groups: Group 1, which was fed normal pellets and did not receive any of the AMUDAM formulation; and Group 2, which was fed normal pellets and received oral administration of 1,000 mg/kg/day of the AMUDAM formulation. The HFD group was split into four groups: Group 3, which was fed a HFD and did not receive any AMUDAM formulation; Group 4, which was fed a HFD and received 50 mg/kg/day of the AMUDAM formulation; Group 5, which was fed a HFD and received 100 mg/kg/day of the AMUDAM formulation; and Group 6, which was fed a HFD and received 200 mg/kg/day of the AMUDAM formulation. (See Table 3). Before the first day of treatment, the animals were fasted overnight, and blood glucose levels were measured at 0, 1, 3, and 6 hours following administration of AMUDAM to check the acute effects of the formulation. (See FIG. 1B).

TABLE 3

| | | Diet | Amudam |
|---|---|---|---|
| Control | Group 1 (Control) (n = 7) | Normal pellet | |
| Control | Group 2 (Treatment-1000) | Normal pellet | 1000 mg/kg |
| | (n = 8) | | |
| HFD | Group 3 (T2DM) (n = 8) | HFD | |
| HFD | Group 4 (Low dose-50) (n = 8) | HFD | 50 mg/kg |
| HFD | Group 5 (Mid dose-100) (n = 8) | HFD | 100 mg/kg |
| HFD | Group 6 (High dose-200) (n = 8) | HFD | 200 mg/kg |

The AMUDAM formulation, which includes various isolated and purified monosaccharides as shown in Table 4, was administered to Groups 2, and 4-6 in different dosage amounts for a period of 4 weeks. During this treatment period, each group continued to receive the normal pellet or HFD pellet throughout the entire study. At the end of the treatment period, the rats were sacrificed 3 hours after Groups 2, and 4-6 received their last dose of the AMUDAM formulation.

TABLE 4

AMUDAM

| | Moles | Wt % |
|---|---|---|
| Galacturonic Acid | 1 | 48.5 |
| Galactose | .46 | 19 |
| Arabinose | .55 | 18.7 |
| Rhamnose | .07 | 2.5 |
| Glucose | .13 | 5.1 |
| Xylose | .08 | 2.8 |
| Mannose | .08 | 3.4 |

For purposes of this study, each of the saccharides used were isolated and purified forms having a purity of at least 90%. L-Rhamnose, L-(+)-Arabinose, D-(+)-Xylose, D-(+)-Mannose, D-(+)-Glucose, D-(+)-Galactose, and D-(+)-Galacturonic acid. However, all isomeric forms of the saccharides are commercially available and contemplated.

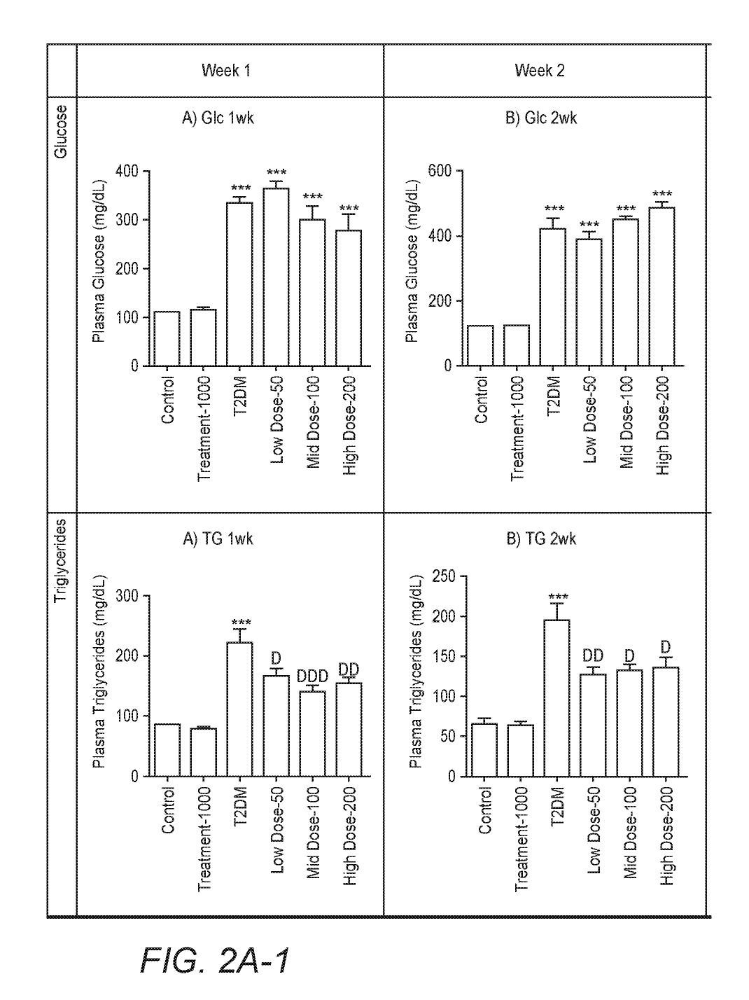

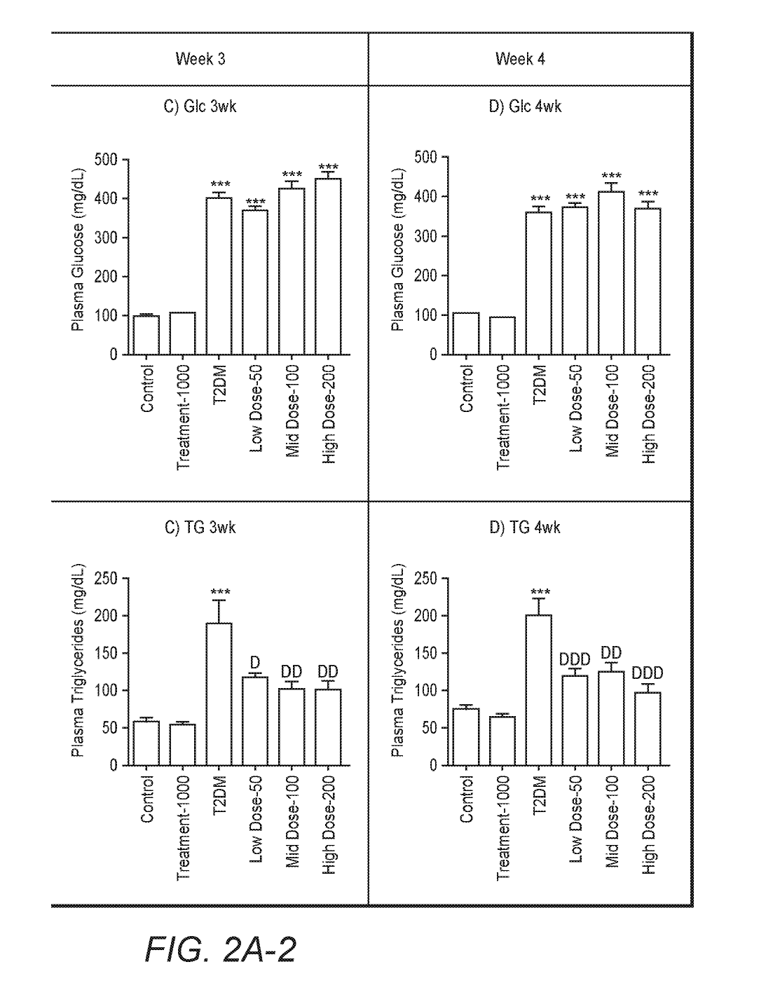

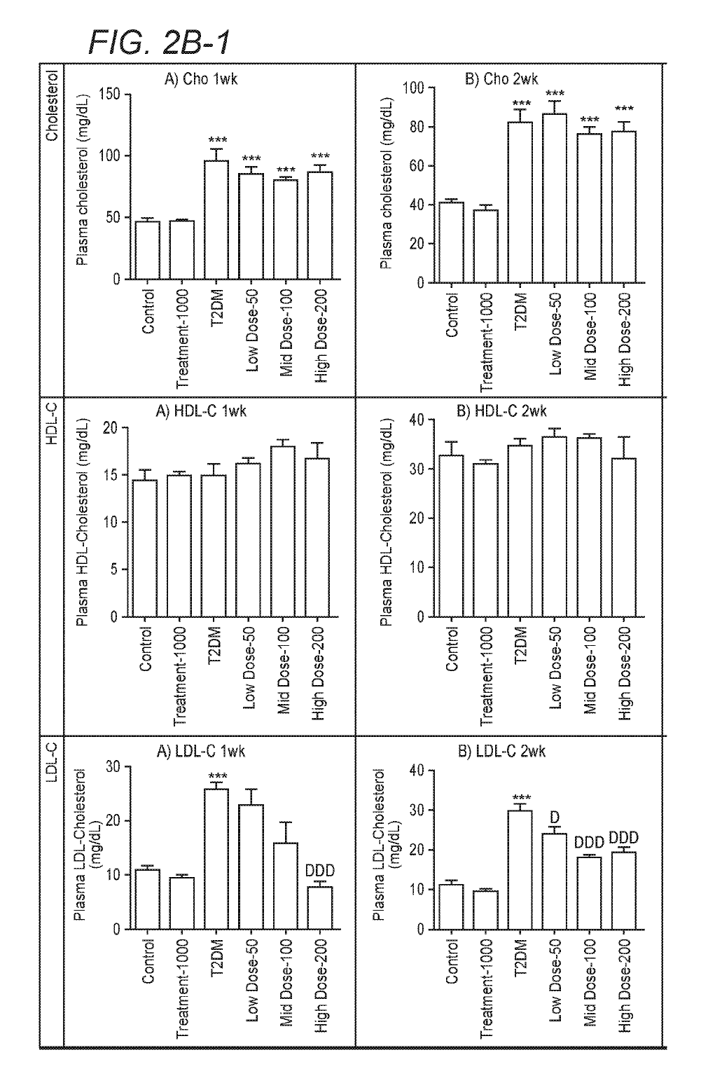

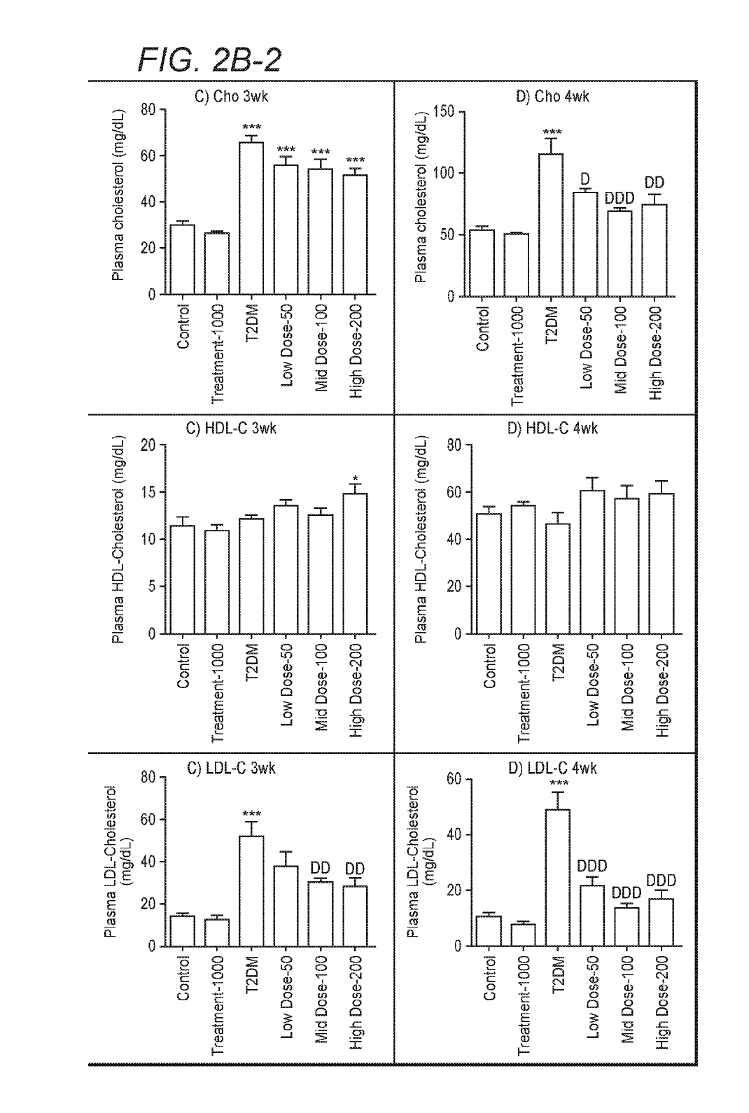

One purpose of Study 1 was to determine the effectiveness of the AMUDAM formulation in reducing biomarkers associated with one or more lipid and liver-related conditions. Another purpose of Study 1 was to determine the effectiveness of the AMUDAM formulation in treating conditions associated with the biomarkers, such as type 2 diabetes. Yet another purpose of Study 1 was to determine whether the AMUDAM formulation would show toxicity or adverse effects when administered in high doses. As clearly shown in FIGS. 2-7 and the accompanying descriptions thereof, the AMUDAM formulation was highly effective in reducing several of the biomarkers, and in significantly reducing fat accumulation in hepatocytes. Additionally, Group 2, which received an administration of 1000 mg/kg/day of the AMUDAM formulation displayed no significant deviations in basal parameters, activity level, feed intake, weight gain, water intake, coagulation time, glucose, triglycerides, cholesterol, high density lipoprotein (HDL), low density lipoprotein (LDL), liver function, oxidative stress markers, morphometry of liver, kidney and histopathology of liver relative to Group 1, which received no AMUDAM formulation. This lack of significant difference indicates the formulation can safely be consumed at high doses without any major side effects.

FIGS. 2A-2B show a comparison of plasma biochemical parameters of the different groups of animals studied at the end of the first, second, third and fourth weeks of the treatment period. With respect to each of glucose, triglycerides, cholesterol and LDL cholesterol, Group 3 (HFD pellets with no AMUDAM) had significantly higher serum levels compared to each of Group 1 (normal pellet and no AMUDAM) and Group 2 (normal pellet and 1,000 mg/kg/day AMUDAM) at all times measured. Groups 4-6, which were fed HFD pellets and given different AMUDAM doses (50, 100, 200 mg/kg body weight/day, respectively) did not show a significant decrease in serum glucose levels during Study 1. However, the hypertriglyceridemia observed in Group 3 animals was reversed by the AMUDAM formulation in Groups 4-6 from the first week of treatment by between 30-40%. This effect persisted until the end of the study, resulting in a total reduction of between 41-53%. Cholesterol levels were also significantly reduced by the AMUDAM formulation, when compared to Group 3 animals. The most significant reduction (between 27-40%) was seen at the end of the four weeks of treatment. Additionally, the LDL cholesterol levels in Group 4-6 animals significantly decreased through the study (between 54-70%), when compared to Group 3 animals.

HDL cholesterol serum levels were slightly lower in Group 3 compared to Groups 1 and 2 at the end of 4 weeks, about 15%. However, Groups 4-6, which were fed HFD pellets and given different AMUDAM doses showed an increase in HDL serum levels (about 10%).

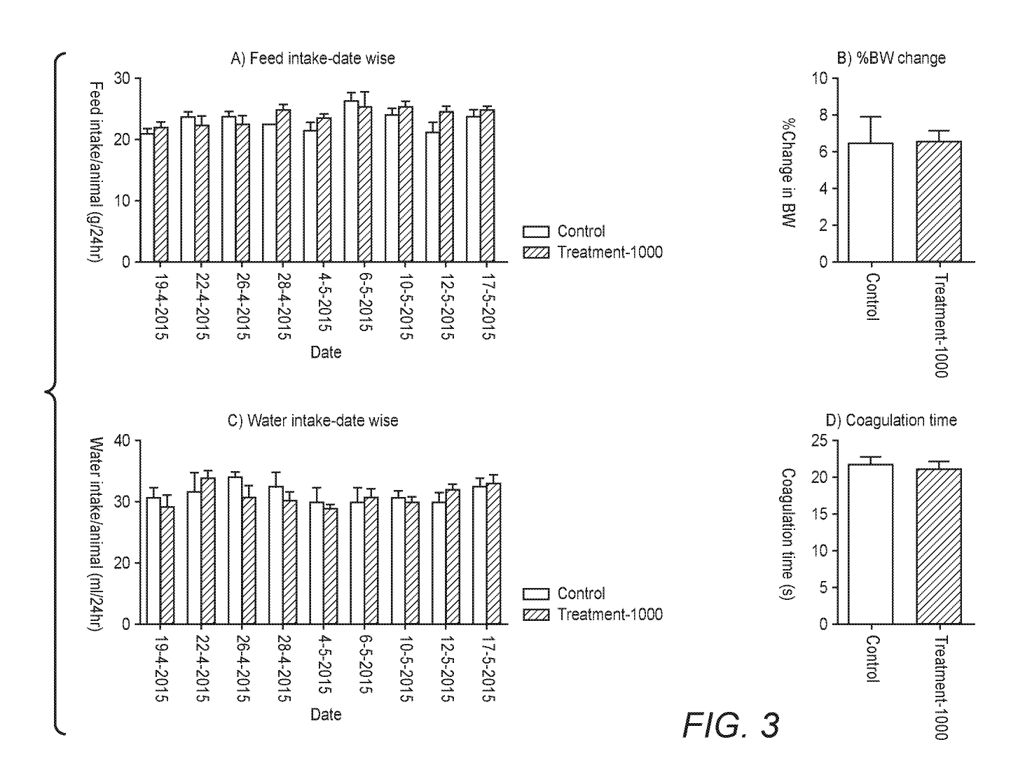

As Groups 5 and 6, receiving the highest and second highest AMUDAM doses of all animals fed HFD pellets, showed the most significant changes in plasma biochemical parameters, it is important to note that there were no significant differences in feed and water intake, body weight change or coagulation time between Groups 1 and 2 throughout the study, as shown in FIG. 3.

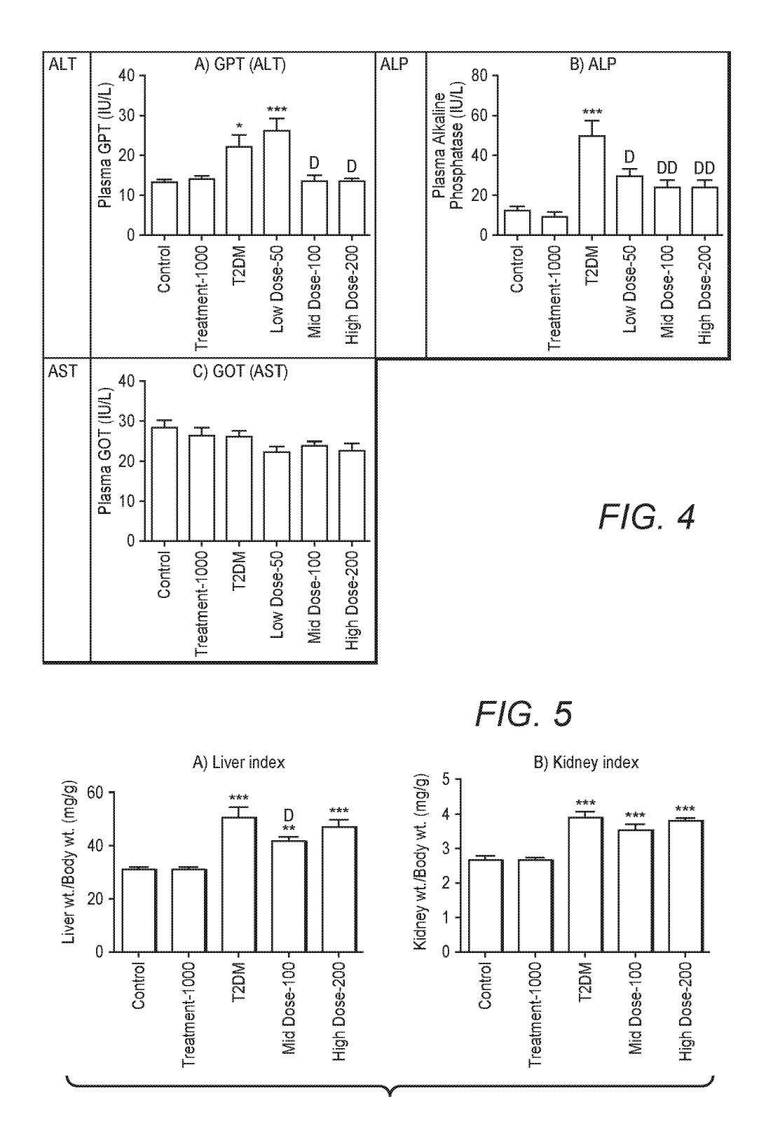

At the end of the 4 week treatment period, liver enzyme tests in serum (aspartate aminotransferase (AST), alanine aminotransferase (ALT), alkaline phosphatase (ALP)) were performed. The results showed that while animals fed a HFD had significantly higher serum ALT and ALP levels compared to animals fed normal pellets, the AMUDAM formulation was effective to significantly decrease serum ALT levels at mid-to-high doses (100 and 200 mg/kg/day, respectively) by between 38-40%, and was effective to significantly decrease serum ALP levels at all doses by 41-53%. (See FIG. 4). There were no significant differences in serum AST levels between the different groups of animals, regardless of whether they were fed normal pellets or HFD pellets.

The morphometry of the liver and kidney of animals in the different groups were also determined at the end of the treatment period. As shown in FIG. 5, there was a significant increase in liver and kidney weight relative to body weight in the Group 3 animals when compared to Groups 1 and 2. 100 mg/kg/day of the AMUDAM formulation significantly reduced the liver index (liver weight/body weight).

3 hours after the last dosing of AMUDAM, the animals were euthanized by CO₂ inhalation, perfused with phosphate buffered saline, and the liver and kidney were excised, weighed, snap frozen in liquid nitrogen, and stored at −80° C. Approximately 100 mg of liver was minced, homogenized, centrifuged at 10,000 g, 4° C. for 10 minutes, and supernatant was collected for the estimation of malonyldialdehyde (MDA), glutathione (GSH), Catalase, superoxide dismutase (SOD) and Glutathione peroxidase (GPx). As shown in FIG. 6, the antioxidant mechanisms of GSH and SOD decreased, and oxidative stress marker MDA increased significantly in the Group 3 rat livers compared to Groups 1 and 2. High doses of the AMUDAM formulation were shown to decrease the MDA level by about 38% compared to Group 3 animals that did not receive the AMUDAM formulation. Glutathione peroxidase and catalase were not significantly altered in any of the groups.

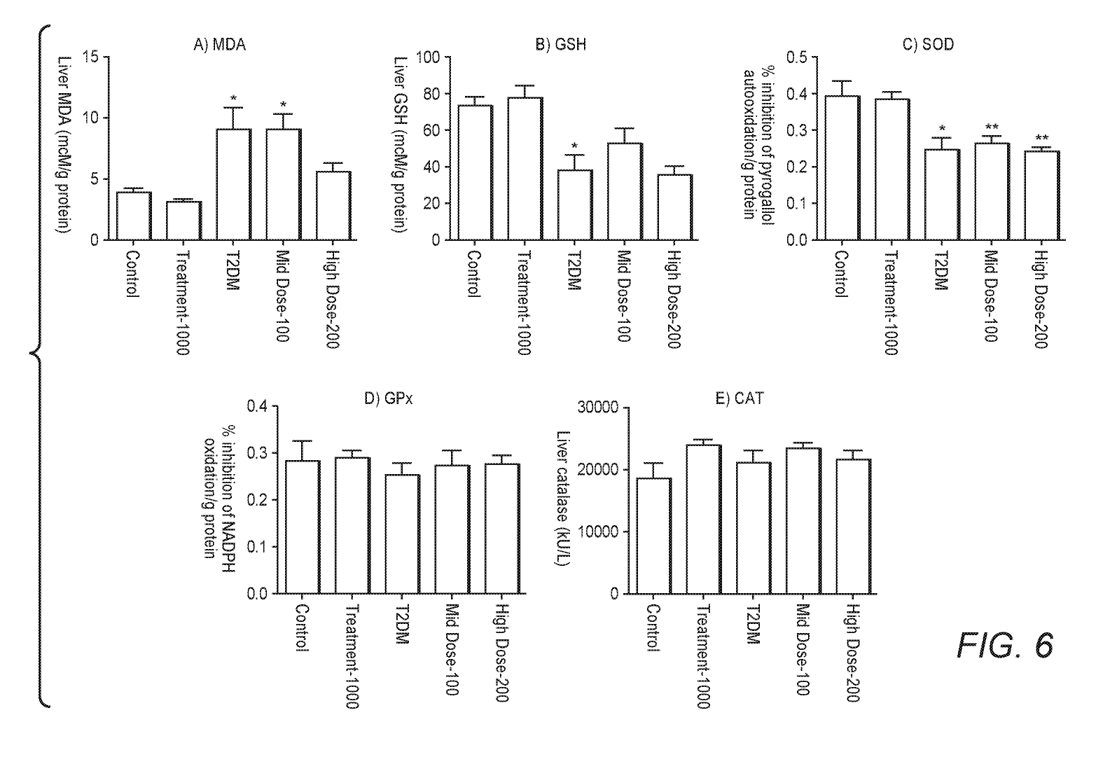

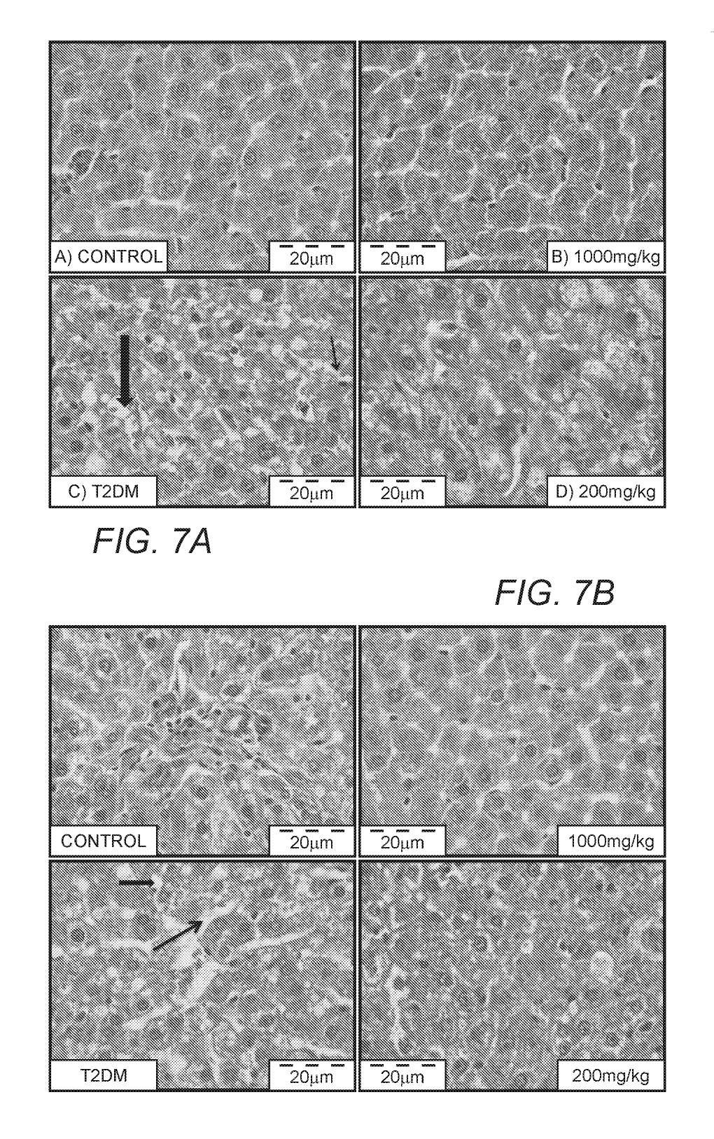

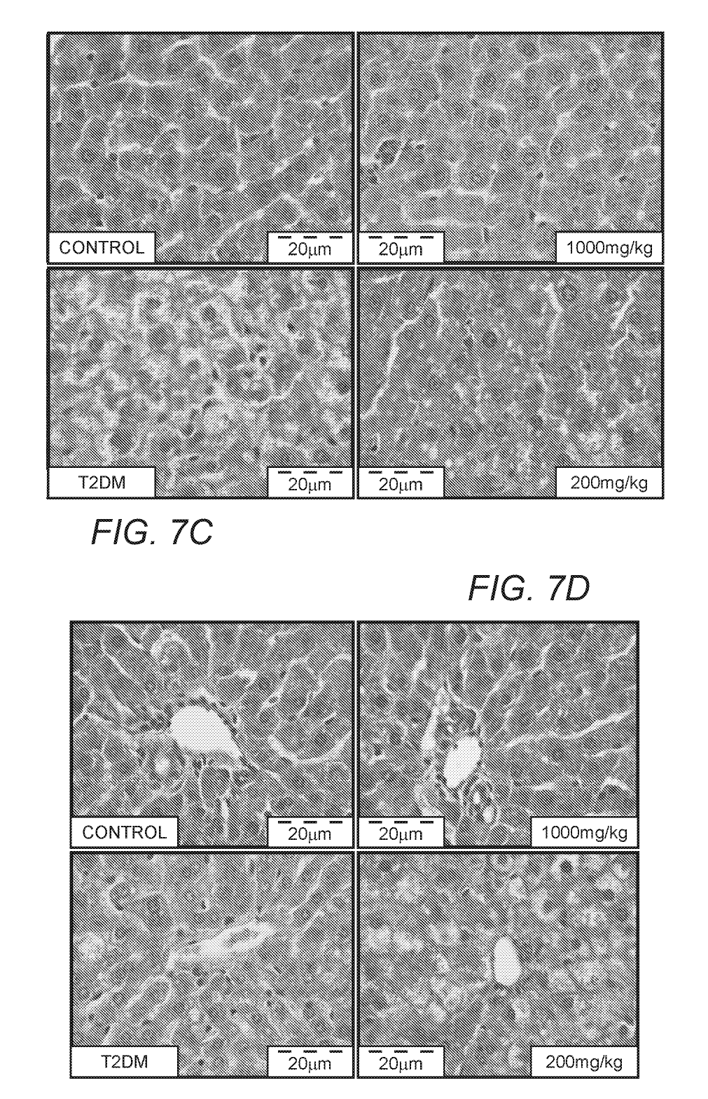

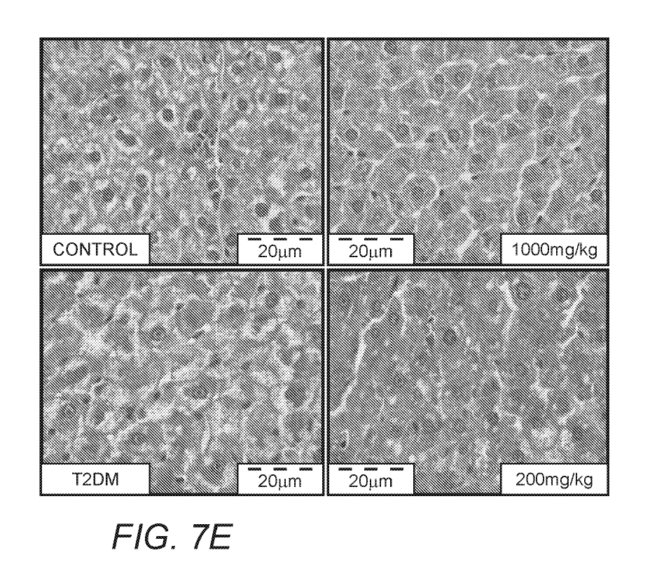

FIGS. 7A-7E illustrate stained sections of liver biopsies from animals in Groups 1 (control group having normal pellets and receiving no formulation), 2 (control group having normal pellets and receiving 1,000 mg/kg/day formulation to assess toxicity of the formulation), 3 (T2DM group having HFD and receiving no formulation), and 6 (group having HFD and receiving 200 mg/kg/day formulation). Group 3 (bottom left) showed that animals fed HFD pellets developed steatosis and hepatic ballooning. The thin arrow points to sinusoidal dilatation in Group 3's liver biopsy. The thick arrow points to fat accumulation in Group 3's liver biopsy. Group 6, which was fed HFD pellets but treated with 200 mg/kg/day AMUDAM, showed improvement in steatosis compared to Group 3, which did not receive the AMUDAM formulation.

To summarize, the results showed a slight increase in HDL cholesterol levels, and significant reduction in triglycerides, cholesterol and LDL cholesterol levels in animals that received the AMUDAM formulation when compared to animals fed a HFD without AMUDAM treatment. Although the HFD clearly caused ALT and ALP levels to significantly rise, 100 mg/kg/day or 200 mg/kg/day of the AMUDAM formulations were able to significantly reduce these levels. Additionally, the AMUDAM formulation was found effective in lowering MDA levels, a biomarker of oxidative stress which is thought to contribute to the aging process, and in reducing the accumulation of fat in the liver.

### Study 2 (VISIVABRM Study)

In Study 2, different subsets and different molar ratios of the saccharides included in the AMUDAM formulation were administered to selected groups of rats at a 200 mg/kg per day to determine the efficacy of different combinations. The NASH group of rats were fed HFD pellets (about 60 w/w %), and 30 w/v % fructose in drinking water (“HFFrD diet”) to induce NASH for ten weeks, and then randomized and treated with different formulations of a “VISIVABRM” for 6 weeks as shown in Table 5. The control rats were fed normal pellets, and not administered any VISIVABRM formulation.

After the 10 weeks of inducing NASH with the HFFrD diet, the NASH group was split into 6 groups, including VISIVABRM 3-7 (Groups 3-7), and Group 2, which received no VISIVABRM treatment. The control group (Group 1) was not split into any further groups. Each of Groups 3-7 received their respective formulations at 200 mg/kg/day for a period of 6 weeks (while continuing the HFFrD diet throughout the entire study), and serum was collected and tested at the end of 2 weeks, and at the end of the 6 week study.

TABLE 5

| | VISIVABRM 3 | | VISIVABRM 4 | | VISIVABRM 5 | | VISIVABRM 6 | | VISIVABRM 7 | |
|---|---|---|---|---|---|---|---|---|---|---|
| | Moles | Wt % | Moles | Wt % | Moles | Wt % | Moles | Wt % | Moles | Wt % |
| Galacturonic | 1 | 48.5 | 1 | 54.7 | 1 | 17.9 | 1 | 12 | 1 | 14.4 |
| Acid | | | | | | | | | | |
| Galactose | .46 | 19 | .46 | 21.4 | 2 | 30.4 | 2 | 20.4 | 2.0 | 24.5 |
| Arabinose | .55 | 18.7 | .55 | 21.1 | 3 | 38 | 6 | 51.1 | 6.0 | 61.1 |
| Rhamnose | .07 | 2.5 | .07 | 2.8 | .36 | 5 | .72 | 6.7 | | |
| Glucose | .13 | 5.1 | | | .10 | 1.5 | .10 | 1 | | |
| Xylose | .08 | 2.8 | | | .45 | 5.7 | .9 | 7.7 | | |
| Mannose | .08 | 3.4 | | | .10 | 1.5 | .10 | 1 | | |

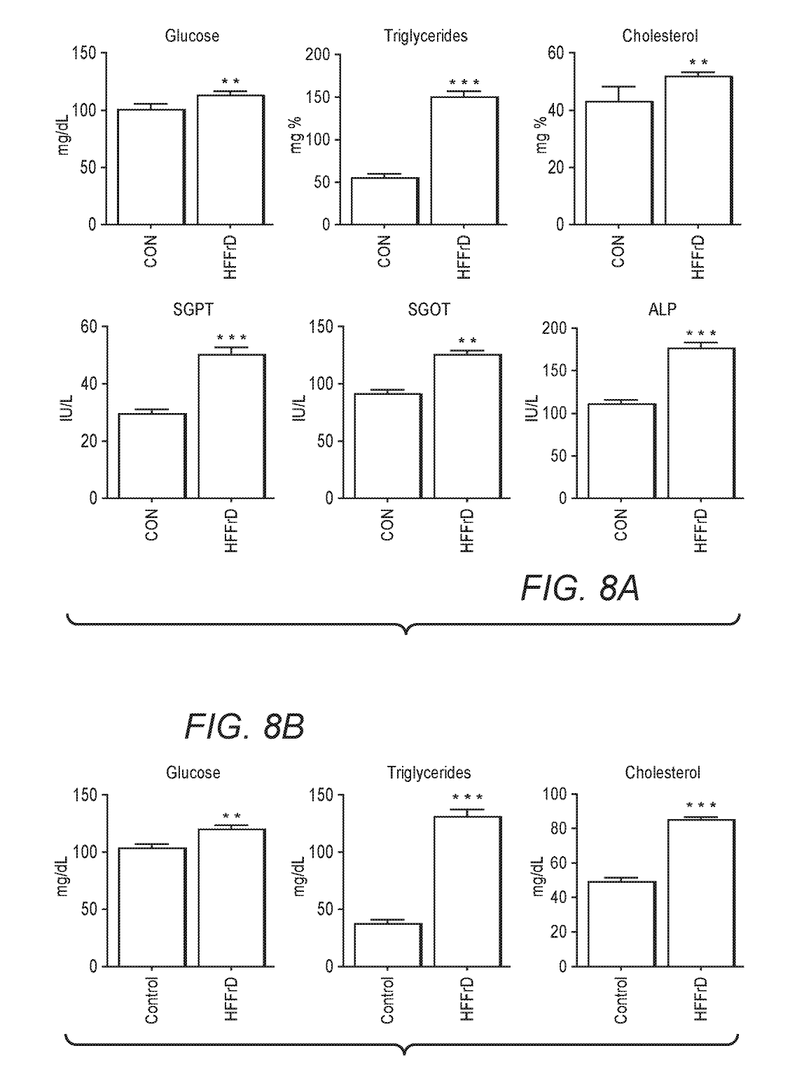

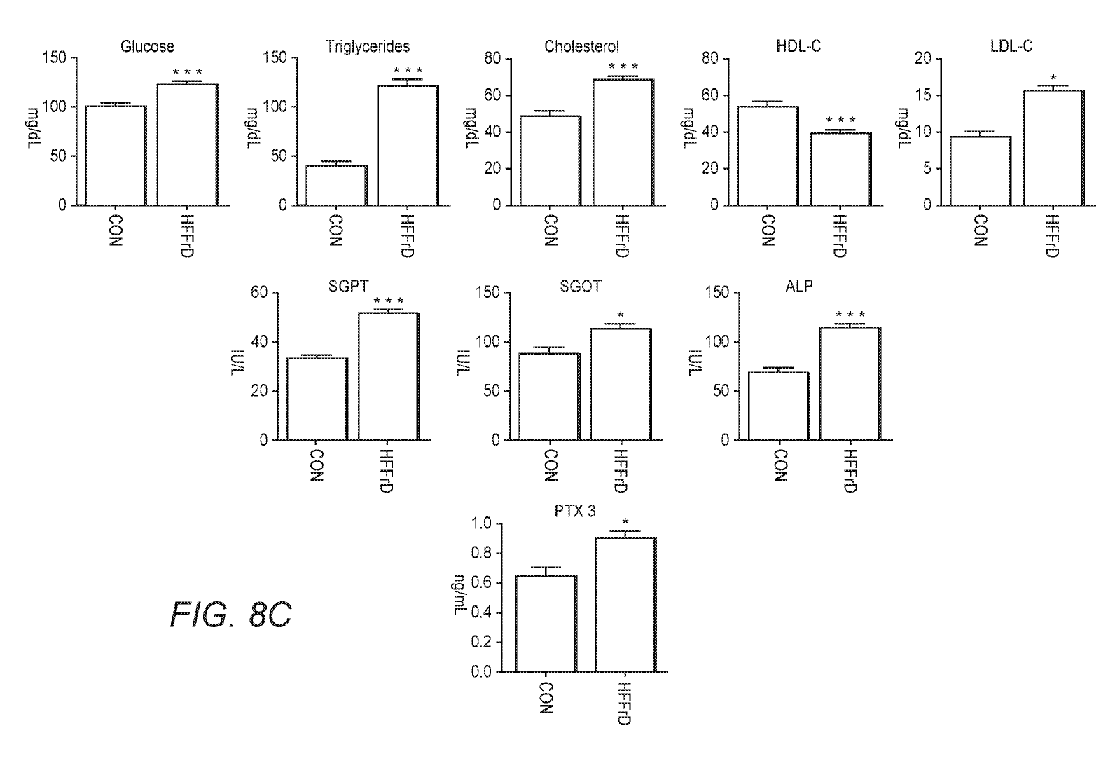

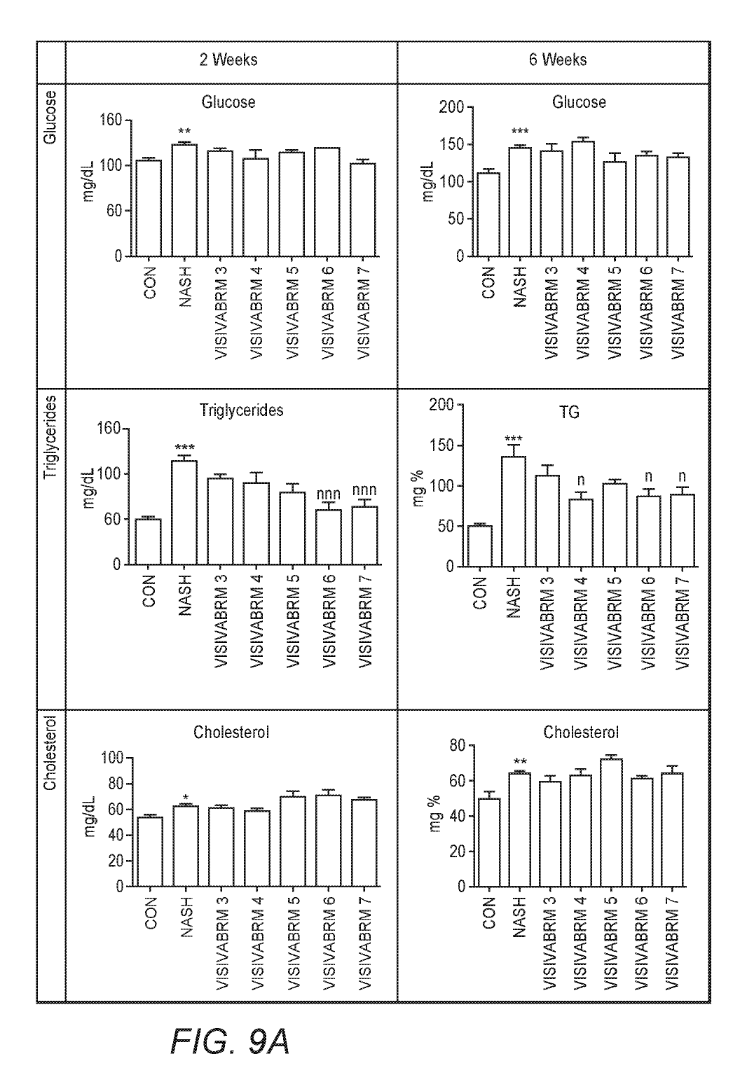

FIG. 8A-C illustrate the serum levels of several biomarkers in the animals during the ten week period prior to administration of a VISIVABRM formulation. FIG. 8A illustrates glucose, triglycerides, cholesterol, SGPT (ALT), SGOT (AST) and ALP levels at 4 weeks, FIG. 8B illustrates glucose, triglycerides and cholesterol levels at 8 weeks, and FIG. 8C illustrates glucose, triglycerides, cholesterol, HDL cholesterol, LDL cholesterol, SGPT (ALT), SGOT (AST), ALP, and pentraxin-related protein (PTX 3) levels at 10 weeks. The animals that were fed the HFFrD diet showed significant increases in glucose, triglycerides, cholesterol, LDL cholesterol, ALT, AST, ALP, and PTX 3 serum levels.

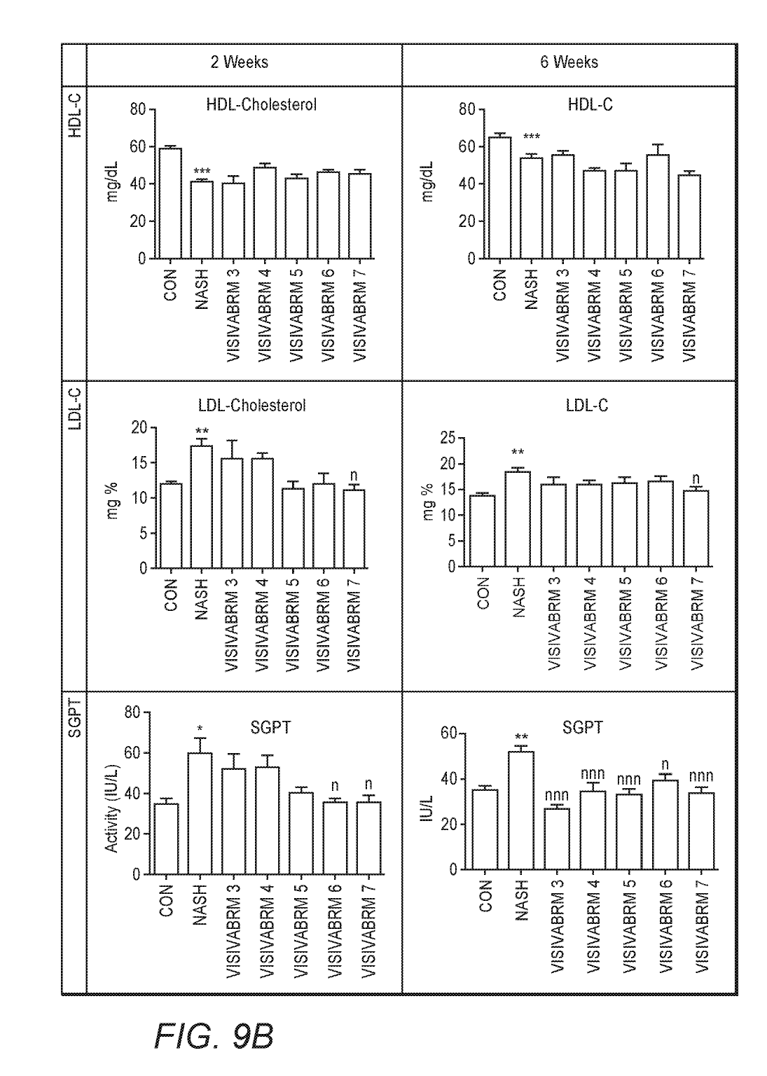

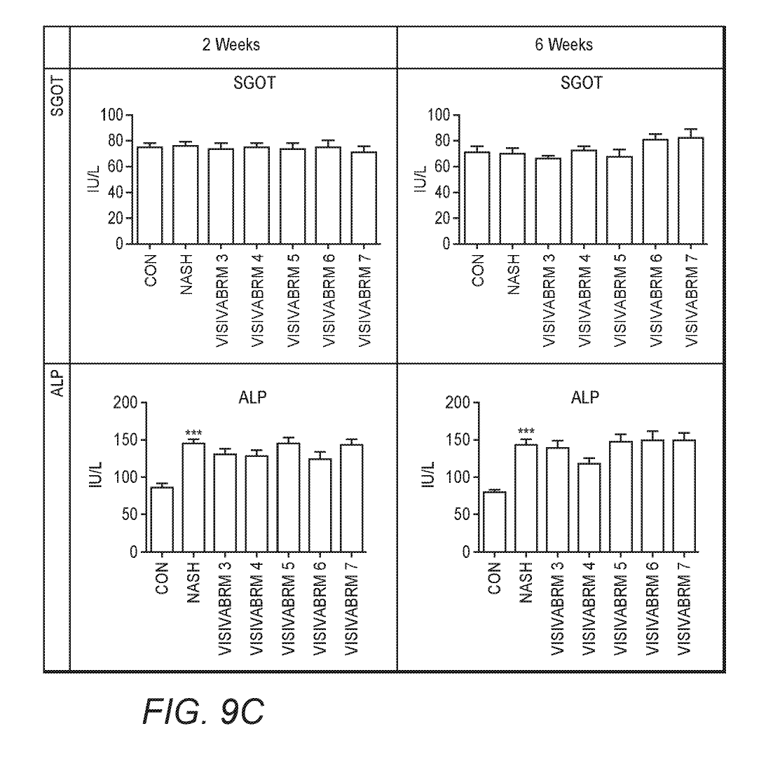

FIGS. 9A-9C show a comparison of plasma biochemical parameters of the different groups of animals studied at the end of the second and sixth weeks of the treatment period. Some of the VISIVABRM formulations were able to lower serum glucose levels in the NASH animals when compared at 2 and 6 weeks to NASH animals not receiving the formulations. Each of the VISIVABRM formulations was highly effective in lowering serum triglyceride levels in the NASH animals when compared at 2 and 6 weeks to NASH animals not receiving the formulations. The formulations reduced serum triglyceride levels between 16-46% at 2 weeks, and between 16-39% at 6 weeks, with VISIVABRM 6 and 7 being the most effective in reversing hypertriglyceridemia at 2 weeks, and VISIVABRM 4, 6 and 7 being the most effective at 6 weeks. Each of the VISIVABRM formulations was effective in reducing serum LDL cholesterol levels in the NASH animals when compared at 2 and 6 weeks to NASH animals not receiving the formulations. The formulations reduced serum LDL levels between 9-35% at 2 weeks, and between 8-20% at 6 weeks, with VISIVABRM 7 having been the most effective formulation in lowering serum LDL cholesterol levels at each of 2 and 6 weeks.

Each of the VISIVABRM formulations was effective in reducing serum SGPT (ALT) levels in the NASH animals when compared at 2 and 6 weeks to NASH animals not receiving the formulations. The formulations reduced serum ALT levels between 13-42% at 2 weeks, and between 21-47% at 6 weeks, with VISIVABRM 3 and 7 appearing to have been the most effective formulations in lowering serum ALT levels at 2 and 6 weeks. Statistically significant reduction is AST or ALP levels were not found, although VISIVABRM-4 showed some reduction in ALP levels at 6 weeks.

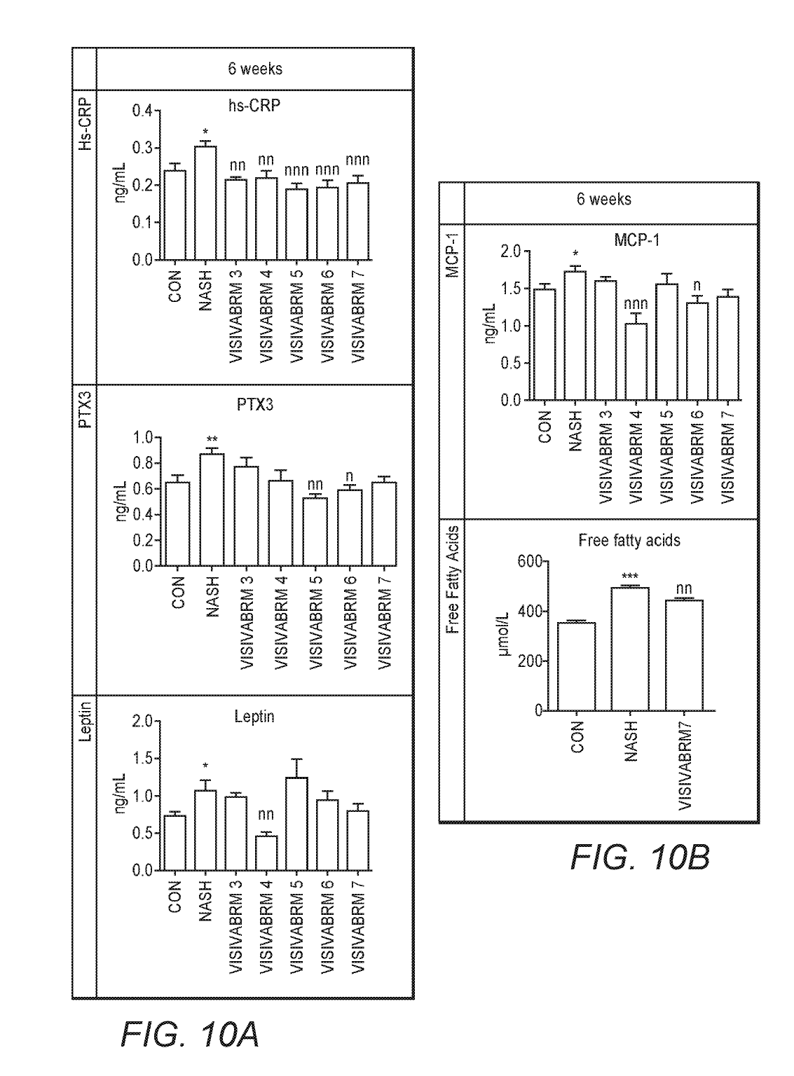

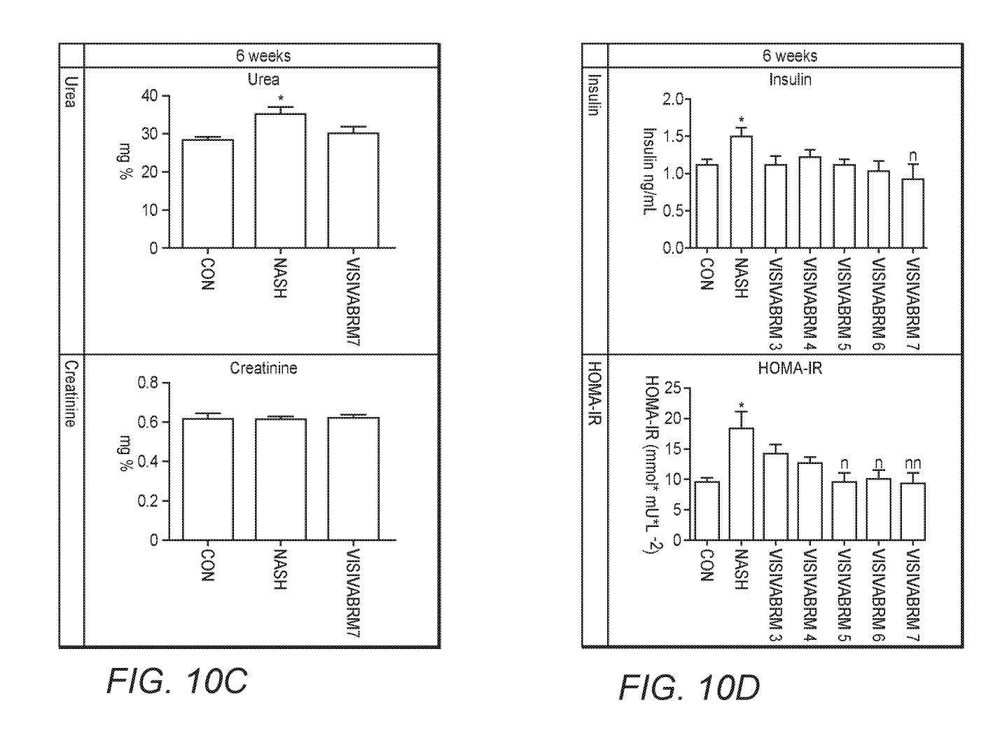

FIGS. 10A-10D show plasma biochemical parameters of the different groups of animals studied at the end of the sixth week of the treatment period. Each of the VISIVABRM formulations was highly effective in lowering serum ultra-sensitive C-reactive protein (hs-CRP) levels in the NASH animals when compared at 6 weeks to NASH animals not receiving the formulations. Each of the formulations reduced serum hs-CRP levels between 28-37% at 6 weeks. Each of the VISIVABRM formulations was also highly effective in lowering serum PTX 3 levels in the NASH animals when compared at 2 and 6 weeks to NASH animals not receiving the formulations. Each of the formulations reduced serum PTX 3 levels between 11-39% at 6 weeks.

With respect to leptin, several of the VISIVABRM formulations were effective to reduce serum leptin levels in NASH animals when compared to NASH animals not receiving the formulations. VISIVABRM 4 was effective in lowering serum leptin levels by about 58%, while VISIVABRM 7 was effective in lowering serum leptin levels by about 27%. Each of the VISIVABRM formulations was also effective in lowering serum monocyte chemoattractant protein-1 (MCP-1) levels in the NASH animals when compared at 2 and 6 weeks to NASH animals not receiving the formulations. Each of the formulations reduced serum MCP-1 levels between 7-40% at 6 weeks.

Free fatty acid and urea levels were each significantly reduced by VISIVABRM 7 formulations in NAHS animals when compared at 6 weeks to NASH animals that did not receive the formulation. Additionally, each of the VISIVABRM formulations were effective to significantly reduce serum insulin and serum HOMA-IR levels (between 18-38% and between 23-51%, respectively) in NASH animals when compared at 6 weeks to NASH animals that did not receive the formulation.

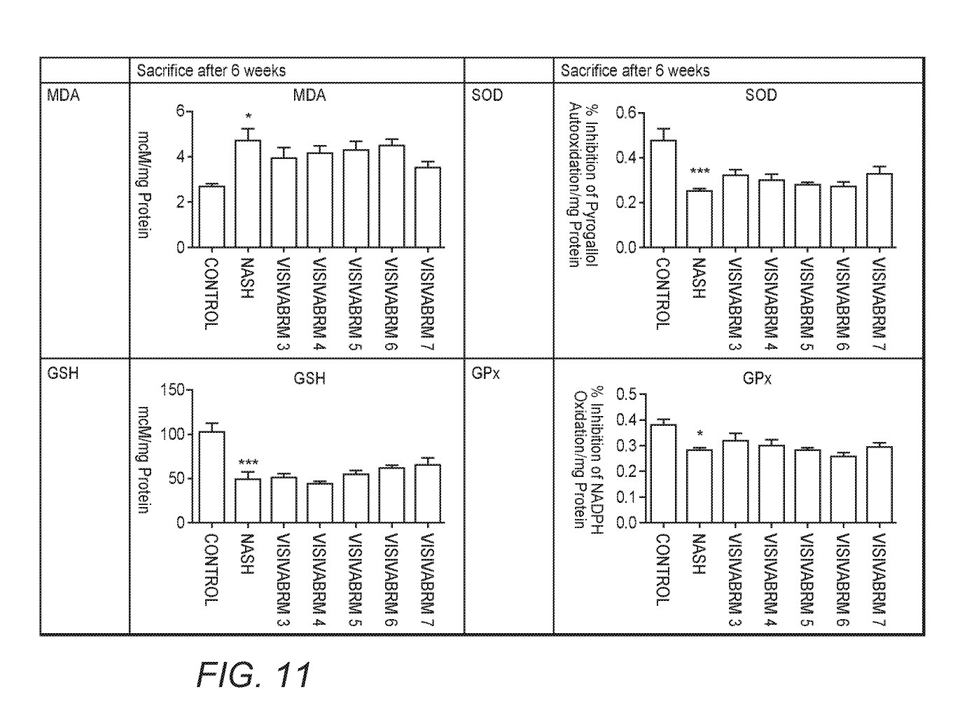

After the 6 week treatment period, the animals were euthanized by CO₂ inhalation, perfused with phosphate buffered saline, and the liver and kidney were excised, weighed, snap frozen in liquid nitrogen, and stored at −80° C. Approximately 100 mg of liver was minced, homogenized, centrifuged at 10,000 g, 4° C. for 10 minutes, and supernatant was collected for the estimation of malonyldialdehyde (MDA), glutathione (GSH), Catalase, superoxide dismutase (SOD) and Glutathione peroxidase (GPX). As shown in FIG. 11, the oxidative stress marker MDA increased significantly in the NASH rat livers compared to the control animals. The VISIVABRM formulations were effective to reduce MDA by between 8-25%.

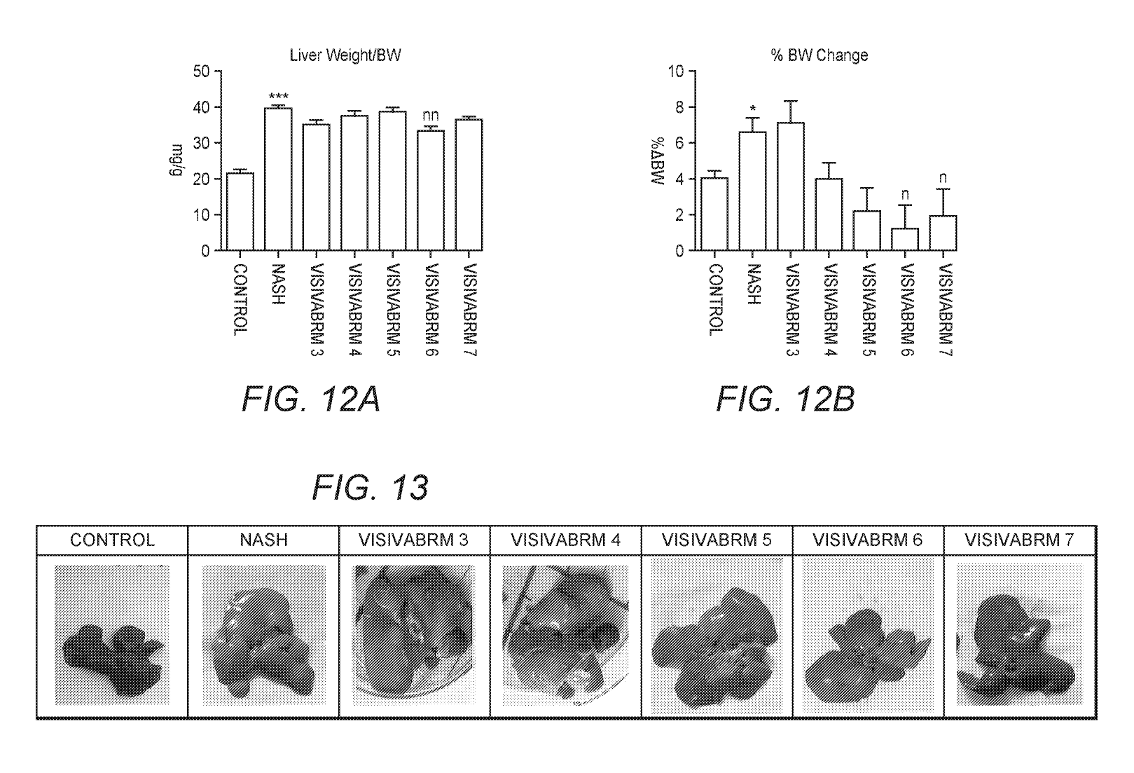

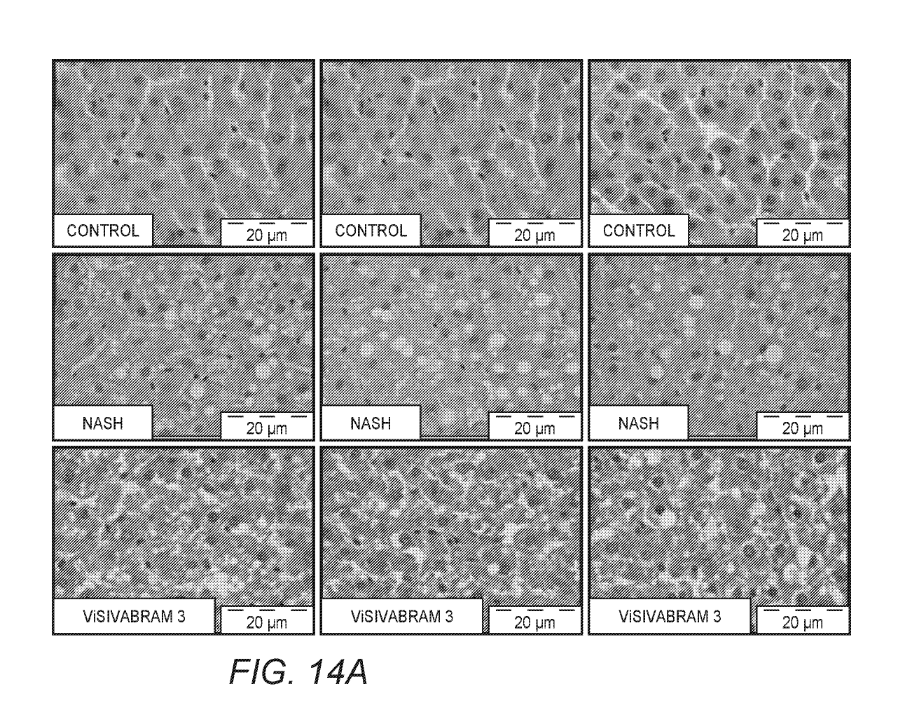

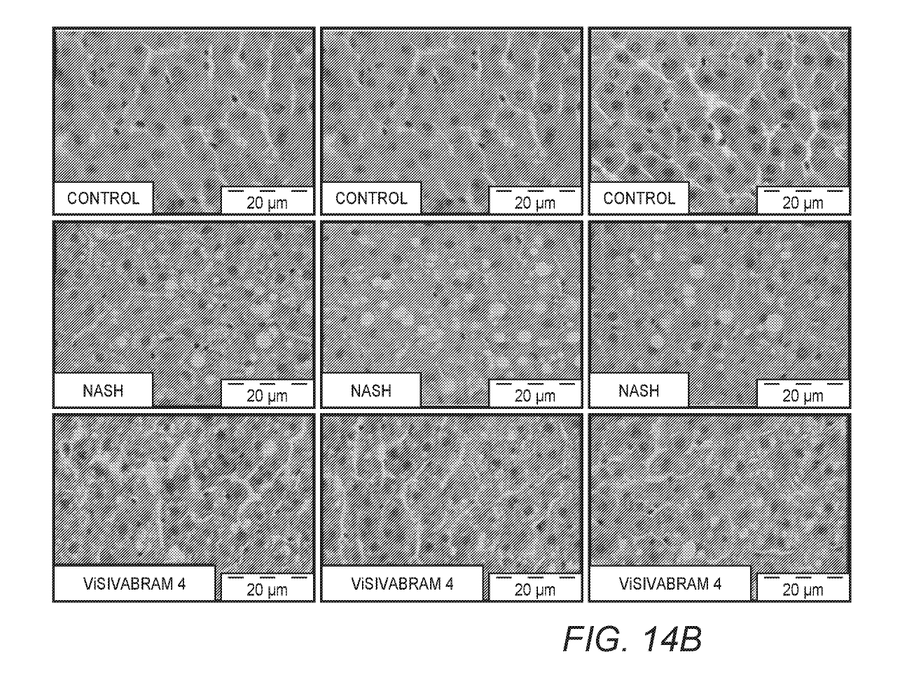

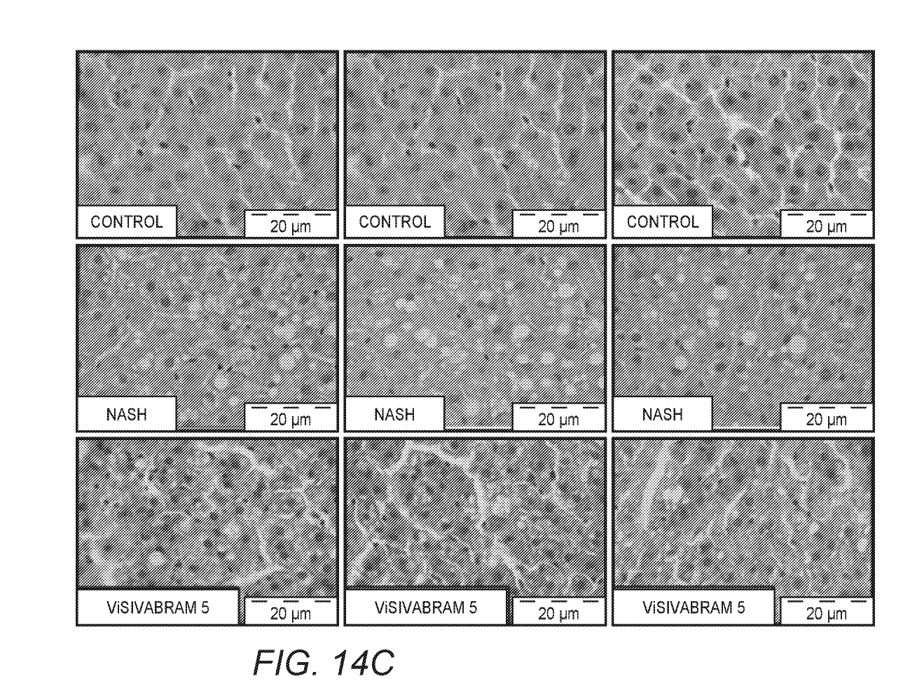

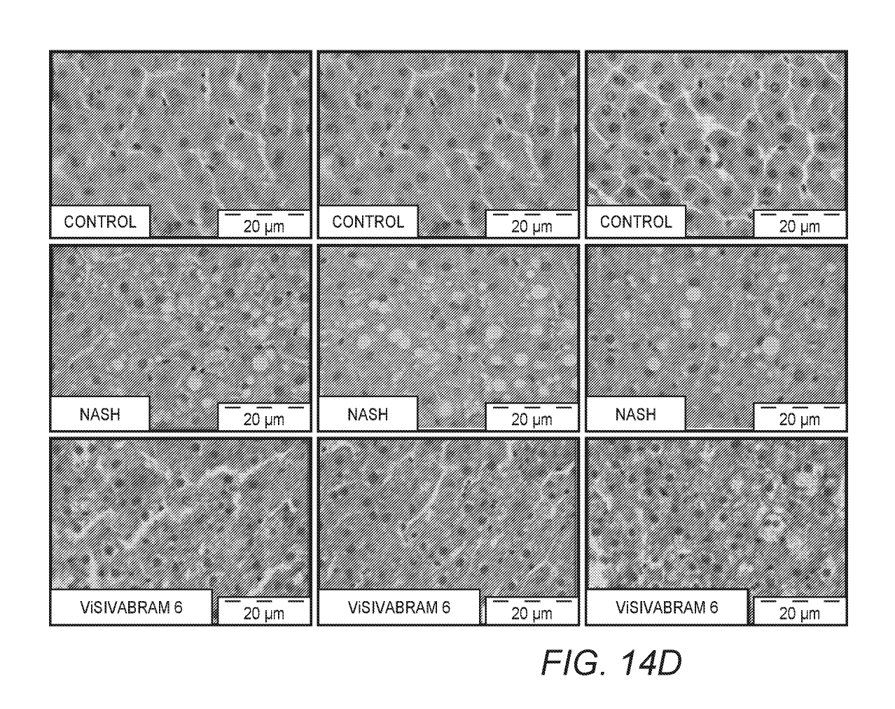

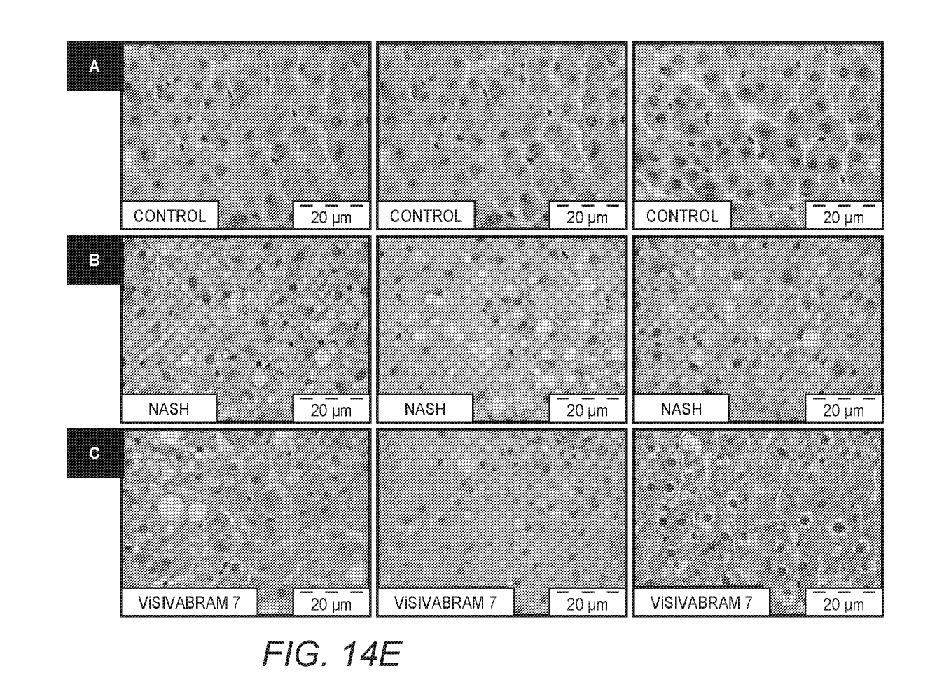

FIG. 12A shows the ratio of liver weight to body weight of animals in the study at the end of the 6 weeks of treatment. FIG. 12B shows the change in body weight of animals in the different groups at the end of the study, with VISIVABRM 4-7 being found effective to significantly reduce the amount of weight gained in NASH animals during the study. FIG. 13 shows images taken of liver samples selected randomly from animals in the different groups. The images were not all taken from the same distance, so the images are not relevant to show a change in size, only a change in color (in color images). FIGS. 14A-14E each show representative photomicrographs of three randomly selected animal livers from each of the control group, NASH group with no treatment, and one of the treated groups (VISIVABRM 3-7). The animal livers were stained with hematoxylin and eosin (H&E). In each of FIGS. 14A-14E, the control showed normal hepatic histoarchitecture, and there was no evidence of sinusoidal dilatation or steatosis. The NASH group (with no treatment) showed evidence of sinusoidal dilatation and steatosis, while VISIVABRM 3-7 were each shown to be at least somewhat effective in improving steatosis. Table 6 is a histological scoring of steatosis in FIG. 14E, showing the level of improvement using the VISIVABRM 7 formulation. The criteria for histological scoring of steatosis is as follows: −, no; +, very less; ++, mild; +++, moderate, +++++, high.

TABLE 6

| Group | Scoring | | |
|---|---|---|---|
| CONTROL | − | − | − |
| NASH | +++ | ++++ | +++ |
| VISIVABRM 7 | ++ | + | ± |

In summary, many of the studied formulations were found to be effective in significantly lowering serum levels or values of several biomarkers associated with lipid and liver-related conditions. The formulation showing the most efficacy with respect to many of the parameters, VISIVABRM 7, was used as a starting point for Applicant's third study in which several combinations of purified and isolated galacturonic acid, galactose and arabinose monosaccharides in different molar proportions were studied.

### Study 3 (BVISIV Study)

In this study, different subsets and different molar ratios of the saccharides included in the VISIVABRM 7 formulation were administered to selected groups of rats at a 200 mg/kg per day to determine the efficacy of different combinations.

During the first 4 weeks of the study, the control group rats were fed a diet of normal pellets, and the high fat diet (HFD) group rats were fed HFD pellets. After the first 4 weeks, the HFD group was injected with streptozotocin (STZ) to induce type 2 diabetes (T2DM).

The control is represented by Group 1, which was fed normal pellets and did not receive any of the BVISIV formulations. The HFD/T2DM groups were separated into 8 groups, and are represented as follows: Group 2, which was fed a HFD and did not receive any BVISIV formulations; Group 3, which was fed a HFD and received 200 mg/kg/day of the BVISIV 3 formulation (same as VISIVABRM 7 formulation); Group 4, which was fed a HFD and received 200 mg/kg/day of the BVISIV 4 formulation; Group 5, which was fed a HFD and received 200 mg/kg/day of the BVISIV 5 formulation; Group 6, which was fed a HFD and received 200 mg/kg/day of the BVISIV 6 formulation; Group 7, which was fed a HFD and received 200 mg/kg/day of the BVISIV 7 formulation; Group 8, which was fed a HFD and received 200 mg/kg/day of the BVISIV 8 formulation; Group 9, which was fed a HFD and received 200 mg/kg/day of the BVISIV 9 formulation.

Before the first day of treatment, the animals were fasted overnight. The BVISIV 3-9 formulations, which include various isolated and purified monosaccharides as shown in Table 7, were administered to Groups 3-9, respectively, for a period of 4 weeks. During this treatment period, each group continued to receive the normal pellet (Group 1) or HFD pellet (Groups 2-9) throughout the entire study. At the end of the treatment period, the rats were sacrificed 3 hours after Groups 3-9 received their last dose of their BVISIV formulation.

TABLE 7

| | BVISIV 3 | | BVISIV 4 | | BVISIV 5 | | BVISIV 6 | | BVISIV 7 | | BVISIV 8 | | BVISIV 9 | |
|---|---|---|---|---|---|---|---|---|---|---|---|---|---|---|
| | MOLES | Wt % | MOLES | Wt % | MOLES | Wt % | MOLES | Wt % | MOLES | Wt % | MOLES | Wt % | MOLES | Wt % |
| Galacturonic | 1 | 14.4 | 1 | 100 | | | | | 1 | 37.1 | 1 | 19.1 | | |
| Acid | | | | | | | | | | | | | | |
| Galactose | 2 | 24.5 | | | 1 | 100 | | | 2 | 62.9 | | | 2 | 28.6 |
| Arabinose | 6 | 61.1 | | | | | 1 | 100 | | | 6 | 80.9 | 6 | 71.4 |

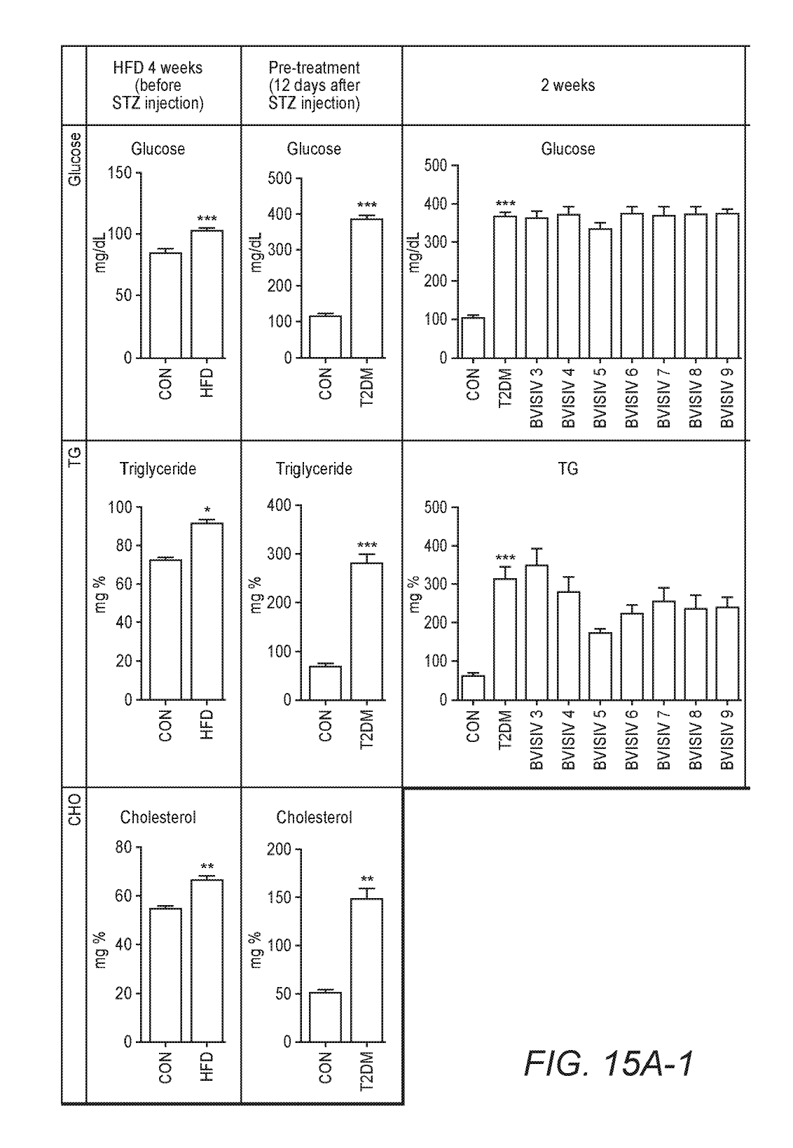

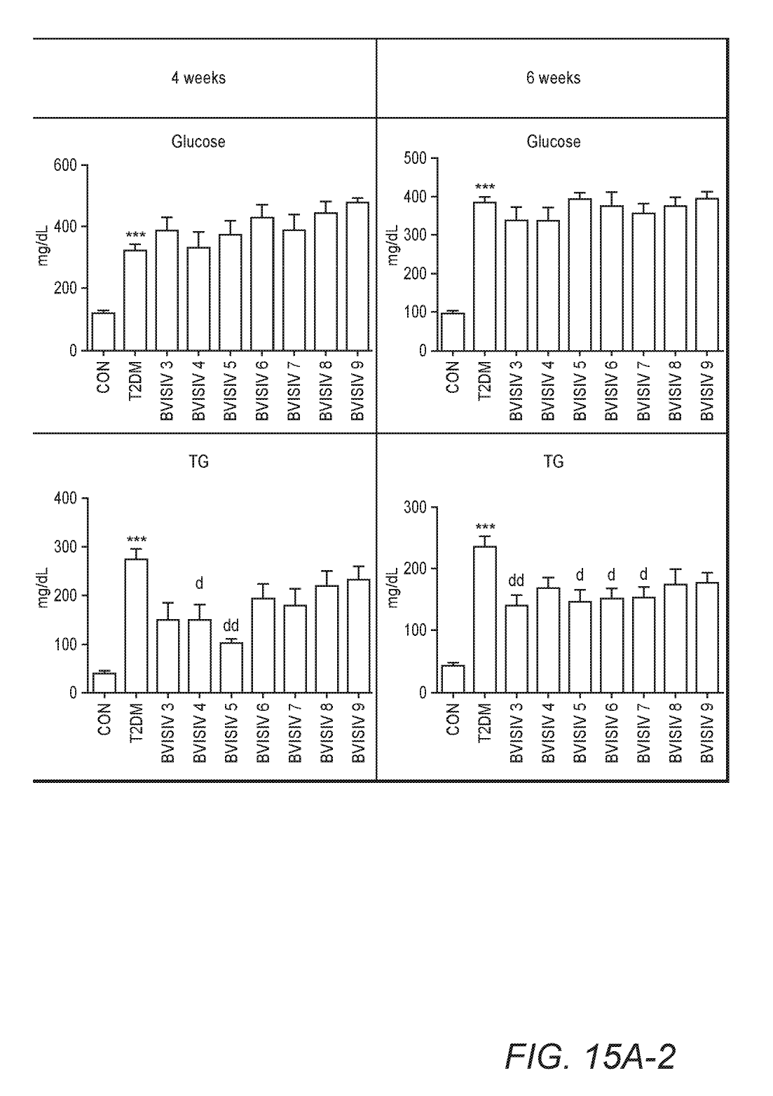

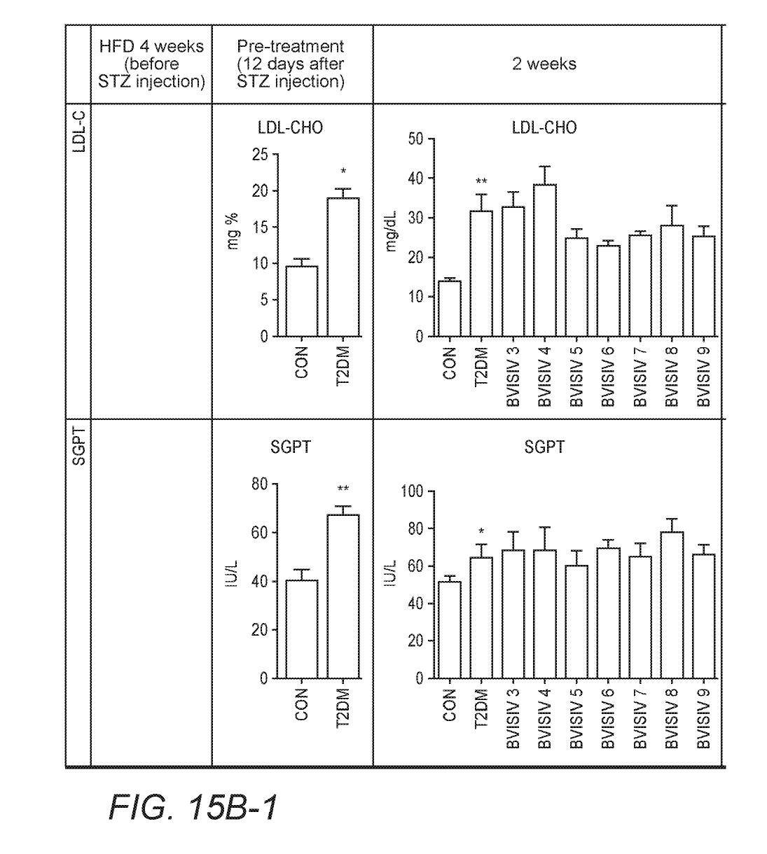

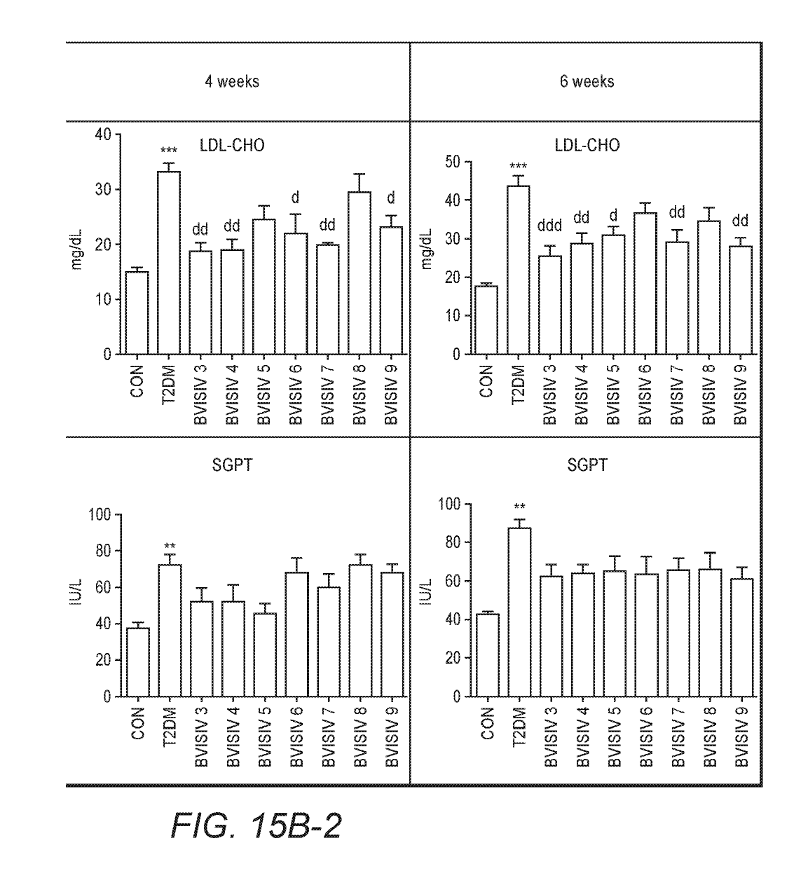

As shown in FIGS. 15A-15B, serum levels of glucose, triglycerides and cholesterol were tested after the first 4 weeks of being fed normal or HFD pellets, and prior to inducing type 2 diabetes in the animals of the HFD group. The results showed that the HFD caused glucose, triglyceride and cholesterol serum levels to increase. About two weeks after injecting the HFD animals with STZ, serum levels of glucose, triglycerides, cholesterol, LDL cholesterol and SGPT (ALT) were tested. The combination of the HFD and the STZ injection caused the HFD animals to become diabetic. The serum levels of each of glucose, triglycerides, cholesterol, LDL and ALT significantly increased in the HFD animals when compared to the control animals.

FIGS. 15A-15B also show a comparison of plasma biochemical parameters of the different groups of animals studied at the end of the second, fourth and sixth weeks of the treatment period. Glucose serum levels were slightly lowered in some of the groups receiving a BVISIV formulation when compared to Group 2 at the end of 6 weeks, with BVISIV 3 and 4 being the most effective.

With respect to triglycerides, LDL cholesterol and ALT, each of the BVISIV formulations were found to be somewhat effective in reducing serum levels of the biomarkers, with each formulation reducing triglyceride levels by between 25-41%, LDL cholesterol levels by between 15-41%, and ALT levels by between 24-30%. Group 3, which received the BVISIV 3 formulation including isolated and purified galacturonic acid, galactose and arabinose, generally outperformed any combination of two saccharides (Groups 7-9). Arabinose, when administered alone or in combination with one of galacturonic acid and galactose did not perform as well as the other formulations, especially with respect to lowering serum LDL and triglyceride levels. However, the combination of galacturonic acid, galactose and arabinose in BVISIV 3 appeared to be more effective than the combination of galacturonic acid and galactose alone.

While galactose alone was found effective to lower triglyceride, LDL and ALT levels, galactose has been reported to cause deterioration of cognitive and motor skills that are similar to symptoms of aging, and is therefore viewed as a model of accelerated aging. Therefore, the liver benefiting effects of galactose may be considered overshadowed or outweighed by its aging effect, and not suitable for administration on a regular basis. However, galacturonic acid can beneficially act to neutralize the aging effects of galactose when administered in combination. Furthermore, some compositions including galacturonic acid and galactose were found to be more effective in reducing triglyceride, LDL and ALT levels than compositions with galactose alone.

In summary, while many formulations having 1, 2 or 3 active components were found to be at least somewhat effective in lowering certain indicators of some lipid and liver-related conditions, the most effective formulations having the least adverse effects is believed to be BVISIV 3, including isolated and purified galacturonic acid monosaccharides, isolated and purified galactose monosaccharides, and isolated and purified arabinose monosaccharides.

All the data in Studies 1-3 was expressed as mean±SEM. Outliers in the raw data, which were identified using Tukey's (Box-and-whiskers plot) method using the interquartile range (IQR), were not included for plotting on the graphs. For statistical significance, means of two groups were compared using Students' t-test, and more than two groups using one-way analysis of variance (ANOVA), followed by Tukey's post hoc test. With respect to significance levels for each of the studies, *P<0.05, **P<0.01, ***P<0.001 vs. Control; ^{D}P<0.05, ^{DD}P<0.01, ^{DDD}P<0.001 vs. T2DM with no formulation; ⁿP<0.05, ⁿⁿP<0.01, ⁿⁿⁿP<0.001 vs. NASH. Plasma and liver tissue samples were analyzed in accordance with Table 8.

TABLE 8

| | Biochemical | |
|---|---|---|
| Parameter | assay/ELISA | Tissue/Plasma |
| Glucose, Triglycerides, Cholesterol, HDL- | Biochemical assay | EDTA-Plasma for all |
| C, LDL-C, GOT = AST, GPT = ALT, ALP, | 1. Accurex Biomedical | assays except for ALP, |
| Creatinine | Pvt. Ltd., Mumbai, | which was done in |
| | India | heparinised plasma |
| | 2. GOT, GPT, ALP: | |
| | Transasia bio- | |
| | medicals Ltd., Solan, | |
| | India | |
| hs-CRP, PTX3, Leptin, fatty acids, MCP-1, | ELISA | EDTA-Plasma |
| insulin | 1. Elabscience | |
| | Biotechnology Co., | |
| | Ltd., WuHan, P.R.C | |
| | 2. Insulin: Crystal | |
| | Chem Inc., IL, USA | |
| MDA, GSH, SOD, GPx, Catalase | Standardized | Liver tissue |
| | biochemical assay using | homogenate |
| | chemicals of Sigma- | |
| | Aldrich, TCI, CDH, SRL, | |
| | LobaChemie, HiMedia, | |
| | Rankem | |

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Also, as used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Moreover, and unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

The discussion herein provides example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

It should be apparent, however, to those skilled in the art that many more modifications besides those already described are possible without departing from the inventive concepts herein. The inventive subject matter, therefore, is not to be restricted except in the spirit of the disclosure. One skilled in the art will recognize many methods and materials similar or equivalent to those described herein, which could be used in the practice of the present invention. Indeed, the present invention is in no way limited to the methods and materials described.

Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

What is claimed is:

1. A composition for lowering at least one of a HOMA-IR value, a total cholesterol serum level, a fat accumulation in liver, a liver oxidative stress marker level, a serum urea level, a serum free fatty acid level, a serum triglyceride level, a serum ALT level, a serum ALP level, a serum hs-CRP level, a serum PTX3 level, a serum leptin level, a serum MCP-1 level, a serum insulin level, and a serum LDL level, comprising:
a set of active components in a therapeutically effective amount, wherein the set of active components consist essentially of at least two isolated and purified saccharides selected from: galacturonic acid, galactose, arabinose, rhamnose, glucose, xylose, and mannose; and
wherein the set of active components are effective, when orally administered, to lower at least one of the HOMA-IR value, the total cholesterol serum level, the fat accumulation in liver, the liver oxidative stress marker level, the serum urea level, the serum free fatty acid level, the triglyceride serum level, the ALT serum level, the ALP serum level, the hs-CRP serum level, the PTX3 serum level, the leptin serum level, the MCP-1 serum level, the insulin serum level, and the LDL serum level.

2. The composition of claim 1, wherein the set of active components consist essentially of isolated and purified galacturonic acid, isolated and purified galactose and isolated and purified arabinose.

3. The composition of claim 2, wherein the molar ratio of galactose to galacturonic acid is between 1 and 3:1, and wherein the molar ratio of arabinose to galacturonic acid is between 4 and 8:1.

4-5. (canceled)

6. The composition of claim 1, wherein the set of active components comprises at least 50 wt % of the composition.

7-12. (canceled)

13. The composition of claim 1, wherein the composition is non-toxic when administered in a rodent model at a dose of 1000 mg/kg/day for a period of four weeks.

14. The composition of claim 1, wherein the set of active components consist essentially of at least three isolated and purified saccharides selected from: galacturonic acid, galactose, arabinose, rhamnose, glucose, xylose, and mannose.

15. The composition of claim 1, wherein the set of active components consist essentially of at least four isolated and purified saccharides selected from: galacturonic acid, galactose, arabinose, rhamnose, glucose, xylose, and mannose.

16-18. (canceled)

19. The composition of claim 1, wherein the set of active components are effective, when orally administered for a period of at least 2 weeks, to lower at least one of the triglyceride serum level, the ALT serum level, the ALP serum level, the hs-CRP serum level, the PTX3 serum level, the leptin serum level, the MCP-1 serum level, the insulin serum level, the HOMA-IR value, the LDL serum level, and the total cholesterol serum level.

20-100. (canceled)

101. A method of lowering at least one of a HOMA-IR value, a total cholesterol serum level, a fat accumulation in liver, a liver oxidative stress marker level, a serum urea level, a serum free fatty acid level, a serum triglyceride level, a serum ALT level, a serum ALP level, a serum hs-CRP level, a serum PTX3 level, a serum leptin level, a serum MCP-1 level, a serum insulin level, and a serum LDL level, comprising:
formulating or obtaining a composition comprising a set of active components in a therapeutically effective amount, wherein the set of active components consist essentially of at least three isolated and purified saccharides selected from: galacturonic acid, galactose, arabinose, rhamnose, glucose, xylose, and mannose; and
administering to a subject in need thereof an effective dose of the composition for a period of at least 6 weeks, wherein the administering results in an at least 10% reduction in the HOMA-IR value, the total cholesterol serum level, the fat accumulation in liver, the liver oxidative stress marker level, the serum urea level, the serum free fatty acid level, the triglyceride serum level, the ALT serum level, the ALP serum level, the hs-CRP serum level, the PTX3 serum level, the leptin serum level, the MCP-1 serum level, the insulin serum level, and the LDL serum level.

102. The method of claim 101, wherein the set of active components consist essentially of isolated and purified galacturonic acid, isolated and purified galactose and isolated and purified arabinose.

103. The method of claim 102, wherein the molar ratio of galactose to galacturonic acid is between 1 and 3:1, and wherein the molar ratio of arabinose to galacturonic acid is between 4 and 8:1.

104-105. (canceled)

106. The method of claim 101, wherein the set of active components comprises at least 50 wt % of the composition.

107. (canceled)

108. The method of claim 101, wherein each of the isolated and purified saccharides has a purity of at least 90% (GC).

109. (canceled)

110. The method of claim 101, wherein the composition is non-toxic when administered in a rodent model at a dose of 1000 mg/kg/day for a period of four weeks.

111. The method of claim 101, wherein the set of active components consist essentially of at least three isolated and purified saccharides selected from: galacturonic acid, galactose, arabinose, rhamnose, glucose, xylose, and mannose.

112. The method of claim 101, wherein the set of active components consist essentially of at least four isolated and purified saccharides selected from: galacturonic acid, galactose, arabinose, rhamnose, glucose, xylose, and mannose.

113. The method of claim 101, wherein the set of active components consist essentially of at least five isolated and purified saccharides selected from: galacturonic acid, galactose, arabinose, rhamnose, glucose, xylose, and mannose.

114. (canceled)

115. The method of claim 101, wherein the isolated and purified saccharides are present in a synergistic amount with respect to lowering at least one of the HOMA-IR value, the total cholesterol serum level, the fat accumulation in liver, the liver oxidative stress marker level, the serum urea level, the serum free fatty acid level, the triglyceride serum level, the ALT serum level, the ALP serum level, the hs-CRP serum level, the PTX3 serum level, the leptin serum level, the MCP-1 serum level, the insulin serum level, and the LDL serum level.

116. The method of claim 101, wherein the subject has at least one of steatohepatitis, obesity and type 2 diabetes.

117. The method of claim 101, wherein the effective dose is between 5-50 mg/kg per day.

118-169. (canceled)
